(19) United States
(12) Patent Application Publication
(10) Pub. No.: US 2025/0000769 A1
(43) Pub. Date: Jan. 2, 2025

(54) SUNSCREEN OR DAILY CARE COMPOSITION COMPRISING BIS-ETHYLHEXYLOXYPHENOL METHOXYPHENYL TRIAZINE AND INORGANIC UV FILTERS

(71) Applicant: BASF SE, Ludwigshafen am Rhein (DE)

(72) Inventors: Myriam SOHN, Grenzach-Wyhlen (DE); Marcel SCHNYDER, Grenzach-Wyhlen (DE); Stanislaw KRUS, Grenzach-Wyhlen (DE)

(21) Appl. No.: 18/689,457

(22) PCT Filed: Sep. 6, 2022
(86) PCT No.: PCT/EP2022/074670

§ 371 Date: Mar. 6, 2024

(30) Foreign Application Priority Data
Sep. 8, 2021 (EP) 21195435.9

(51) Int. Cl. A61K 8/49; A61K 8/27; A61K 8/29; A61Q 17/04

(52) U.S. Cl. CPC A61K 8/4966; A61K 8/27; A61K 8/29; A61Q 17/04

(57) ABSTRACT

The present application relates to a sunscreen or daily care composition comprising bis-ethylhexyloxyphenol methoxyphenyl triazine (INCI bis-ethylhexyloxyphenol methoxyphenyl triazine), titanium dioxide, and zinc oxide.

The present application relates to a sunscreen or daily care composition comprising bis-ethylhexyloxyphenol methoxyphenyl triazine (INCI bis-ethylhexyloxyphenol methoxyphenyl triazine, also known as BEMT also known as Bemotrizinol), titanium dioxide (TiO2), and zinc oxide (ZnO).

UV radiation causes harmful effects on the human skin. Beside the acute effect of sunburn of the skin, UV radiation is also known to increase the risk of skin cancer. Furthermore, long time exposure to UV-A and UV-B light can cause phototoxic and photo allergenic reactions on the skin and can accelerate skin aging.

To protect the human skin from UV radiation, various sun protecting UV filters (also referred to as UV absorbers) exist including UV-A filter, UV-B filter and broadband filters. These filters are added to sunscreen or daily care compositions. The UV filters are either organic or inorganic, particulate or non-particulate compounds, of which all have a high absorption efficacy in the UV-light range. In general, UV light can be divided into UV-A radiation and UV-B radiation. Depending on the position of the absorption maxima, UV-filters are divided into UV-A and UV-B filters. In case an UV-filter absorbs both, UV-A and UV-B light, it is referred to as a broadband absorber. Since broadband UV filters provide protection against UVA and UVB radiation it is preferred to add these kind of UV filters into sunscreen or daily care composition. However, there is still the need of improved formulations that provide efficient and safe UV protection. Particularly efficient UV filter-containing compositions provide a high SPF value whereas particularly safe UV filter-containing compositions comprise as little UV filter as possible in order to minimize the risk of any potential allergic reaction. Further, particularly efficient UV filter also are photostable. UV filter system should not photodegrade, since photoinstable UV filter mixture can produce unknown photodegradation product or free radicals which can lead to adverse effects such as skin irritation and instability of the sunscreen.

Regarding customer requirements, it appears that the sunscreen or daily care compositions having a reduced stickiness, reduced whitening effect when applied, and reduced fabric staining before and/or after washing are preferred. In addition, even if main marketed products are providing both UVB and UVA protection according to the current standards, there is a need for improvement of the water resistance. Water resistance is an important performance indicator for end consumers, since users exemplarily need UV protection while swimming or during outdoor activities in summer (sweating), but is yet difficult to be achieved.

One suitable broadband UV filter is bis-ethylhexyloxyphenol methoxyphenyl triazine (INCI bis-ethylhexyloxyphenol methoxyphenyl triazine, BEMT) having the formula (1).

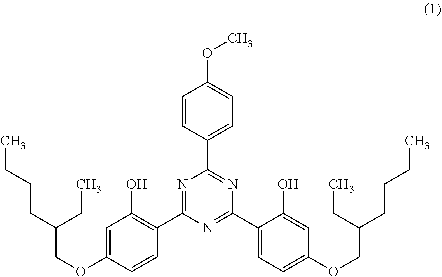 [SMILES: CCCCC(CC)COc1ccc(-c2nc(-c3ccc(OC)cc3)nc(-c3ccc(OCC(CC)CCCC)cc3O)n2)c(O)c1]

Against this background, there is an ongoing need for sunscreen or daily care compositions for the daily use, which provide efficient sun protection and meet the consumer's demands with respect to protection performance and convenience upon and after applying the product. In particular, it was an object of the present invention to provide efficient and photostable sunscreen or daily care compositions. It was a further object of the present invention to provide efficient and safe sunscreen or daily care compositions perfectly meeting the consumer's demands with respect to protection performance and convenience upon and after applying the product. Further, it was an object of the present invention to provide sunscreen or daily care compositions, which are free of certain UV filters under discussion. In particular, it was an object of the present invention to provide a sunscreen or daily care compositions having an improved water resistance. In addition, it was an object of the present invention to provide a microbiome friendly sunscreen or daily care composition.

It has surprisingly been found that at least one of these objects can be achieved by the sunscreen or daily care composition according to the present invention.

In particular, the inventors of the present application found that the sunscreen or daily care composition according to the present invention provides an efficient and safe UV-A and UV-B protection with e.g. a reduced UV filter amount. Further, the inventors of the present application found that the sunscreen or daily care composition according to the present invention are particularly convenient for the user since they provide a reduced stickiness, and/or reduced fabric staining before and/or after washing, and/or reduced formulation yellowing, and/or reduced gloss (of the applied formulation; also referred to as shininess), and/or improved film homogeneity. In addition, the inventors of the present application found that the sunscreen or daily care composition according to the present invention provides an efficient UV-A and UV-B protection nevertheless the composition is free of certain UV filter under discussion, i.e. octocrylene, benzophenone-3, ethylhexyl methoxycinnamate, and homosalate.

Hence, according to a first aspect A, the present invention relates to a sunscreen or daily care composition comprising
(i) bis-ethylhexyloxyphenol methoxyphenyl triazine (INCI bis-ethylhexyloxyphenol methoxyphenyl triazine);
(ii) titanium dioxide; and
(iii) zinc oxide,
   wherein the sunscreen or daily care composition is free of, 4,4′,4″-(1,3,5-triazine-2,4,6-triyltriimino)tris-benzoic acid-tris(2-ethylhexyl)ester (INCI ethylhexyl triazone, also known as octyl triazone, also known as EHT), hexyl 2-[4-(diethylamino)-2-hydroxybenzoyl]benzoate (INCI diethylamino hydroxybenzoyl hexyl benzoate, also known as DHHB), 2,2′-Methylenbis[6-(2H-benzotriazol-2-yl)-4-(1,1,3,3-tetramethylbutyl)phenol](INCI Methylene Bis-Benzotriazolyl Tetramethylbutylphenol, also known as bisoctrizole, also known as MBBT), and butyloctyl salicylate and wherein if the sunscreen or daily care composition further comprises (RS)-2-Ethylhexyl (2E)-3-(4-methoxyphenyl)prop-2-enoate (INCI ethylhexyl methoxycinnamate, also known as octyl ethoxycinnamate, also known as octinoxate, also known as OMC) said sunscreen or daily care composition is free of 1-(4-(1,1-dimethylethyl)phenyl)-3-(4-methoxyphenyl)propane-1,3-dione (also known as avobenzone, also known as BMDBM).

In the following, preferred embodiments of the above sunscreen or daily care composition are described in further detail. It is to be understood that each preferred embodiment is relevant on its own as well as in combination with other preferred embodiments.

In a preferred embodiment A1 of the first aspect, the titanium dioxide has a number-average elementary particle diameter of less than 1000 nm, preferably of less than 100 nm, and in particular less than 50 nm.

In a preferred embodiment A2 of the first aspect, the zinc oxide has a number-average elementary particle diameter of less than 1000 nm, preferably of less than 200 nm, and in particular less than 150 nm.

In a preferred embodiment A3 of the first aspect, the titanium dioxide is coated, preferably wherein the titanium dioxide is a coated titanium dioxide which is a pigment that has undergone one or more surface treatments of chemical, electronic, mechanochemical and/or mechanical nature preferably with compounds selected from the group consisting of amino acids, beeswax, fatty acids, fatty alcohols, anionic surfactants, lecithins, sodium, potassium, zinc, iron or aluminium salts of fatty acids, metal alkoxides (of titanium or aluminium), polyethylene, silicones, proteins (collagen, elastin), alkanolamines, silicon oxides, metal oxides, sodium hexametaphosphate, silica and mixtures thereof, more preferably wherein the titanium oxide is coated with aluminium hydroxide, stearic acid, and mixtures thereof. In a further preferred embodiment, the titanium oxide is coated with silica, dimethicone, and mixtures thereof.

In a preferred embodiment A4 of the first aspect, the zinc oxide is uncoated or coated, preferably wherein the zinc oxide is coated with inorganic surface coatings selected from the group consisting of aluminum oxide (Al₂O₃), aluminum hydroxide Al(OH)₃, aluminum oxide hydrate, sodium hexametaphosphate (NaPO₃), sodium meta-phosphate (NaPO₃)ₙ, silicon dioxide (SiO₂), iron oxide (Fe₂O₃), and mixtures thereof; or organic surface coatings selected from the group consisting of vegetable or animal aluminum stearate, fatty acids, dimethylpolysiloxane, methylpolysiloxane, simethicone, triethoxycaprylylsilane, octyltrimethoxysilane, and mixtures thereof; or mixtures of inorganic surface coatings with organic surface coatings, more preferably triethoxycaprylylsilane.

In a preferred embodiment A5 of the first aspect, the weight ratio of the sum of titanium dioxide and zinc oxide to the bis-ethylhexyloxyphenol methoxyphenyl triazine is in the range of 1:1 to 20:1, preferably 1:1 to 12:1, and in particular 2:1 to 6:1 and/or wherein the weight ratio of the titanium dioxide to the zinc oxide is in the range of 4:1 to 1:3, preferably of 3:1 to 1:2, more preferably of 5:2 to 1:1, and in particular of 2:1 to 6:5.

In a preferred embodiment A6 of the first aspect, the bis-ethylhexyloxyphenol methoxyphenyl triazine (INCI bis-ethylhexyloxyphenol methoxyphenyl triazine) is added in the oil phase.

In a preferred embodiment A7 of the first aspect, the sunscreen or daily care composition comprises at least one further, different UV filter.

In a preferred embodiment A8 of the first aspect, the sunscreen or daily care composition does not comprise a further, different UV filter.

In a preferred embodiment A9 of the first aspect, the sunscreen or daily care composition comprises at least one compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, a fused ring cyanoacrylate derivative, polyester-25, and combinations thereof.

In a preferred embodiment A10 of the first aspect, the titanium dioxide and zinc oxide are comprised in an amount of 1 to 35 wt.-%, preferably of 5 to 25 wt.-%, and in particular of 8 to 20 wt.-%, based on the total amount of the sunscreen or daily care composition.

In a preferred embodiment A11 of the first aspect, the sunscreen or daily care composition comprises
(i) bis-ethylhexyloxyphenol methoxyphenyl triazine (INCI bis-ethylhexyloxyphenol methoxyphenyl triazine) in an amount of 1 to 10 wt.-%, preferably of 1.5 to 6 wt.-%;
(ii) titanium dioxide in an amount of 2 to 20 wt.-%, preferably of 4 to 15 wt.-%; and
(iii) zinc oxide in an amount of 1 to 20 wt.-%, preferably of 3 to 15 wt.-%; each based on the total weight of the sunscreen or daily care composition.

In a preferred embodiment A12 of the first aspect, the sunscreen or daily care composition comprises a total of 5 to 40 wt.-% of UV filters, preferably of 9 to 30 wt.-%, and in particular of 9 to 20 wt.-%, based on the total weight of the sunscreen or daily care composition.

In a preferred embodiment A13 of the first aspect, the sunscreen or daily care composition provides a SPF of more than 15, preferably of more than 30 or a SPF of more than 50, preferably of more than 60, or a SPF of more than 90, preferably of more than 100.

In a preferred embodiment A14 of the first aspect, the sunscreen or daily care composition provides a higher SPF value than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to embodiments A to A13.

In a preferred embodiment A15 of the first aspect, the sunscreen or daily care composition provides a photostability of more than 85%, preferably of more than 90%, and in particular of more than 95%.

In a preferred embodiment A16 of the first aspect, the sunscreen or daily care composition provides a higher photostability than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to embodiments A to A15.

In a second aspect B, the present invention relates to a method of enhancing the photostability, and/or reducing the stickiness, and/or improving the water resistance, and/or improving the film homogeneity, and/or reducing fabric staining before and/or after washing, and/or reducing the gloss, and/or reducing the formulation yellowing, and/or improving the compatibility with the microbiome, preferably the human skin microbiome, of a sunscreen or daily care composition comprising titanium dioxide and zinc oxide, preferably the sunscreen or daily care composition according to aspect A and all its embodiments, by applying bis-ethylhexyloxyphenol methoxyphenyl triazine, wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol] (bisoctrizole), and butyloctyl salicylate and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dione (avobenzone).

In this connection, the present invention also relates to the use of bis-ethylhexyloxyphenol methoxyphenyl triazine in a sunscreen or daily care composition comprising titanium dioxide and zinc oxide, for enhancing the photostability and/or for reducing the stickiness and/or for improving the water resistance, and/or for improving the film homogeneity, and/or reducing fabric staining before and/or after washing, and/or reducing the gloss, and/or reducing the formulation yellowing, and/or for improving the compatibility with the microbiome, preferably the human skin microbiome, of said sunscreen or daily care composition, preferably according to aspect A and all its embodiments.

In a third aspect, the present invention relates to a sunscreen or daily care composition comprising
(i) bis-ethylhexyloxyphenol methoxyphenyl triazine (INCI bis-ethylhexyloxyphenol methoxyphenyl triazine);
(ii) titanium dioxide; and
(iii) zinc oxide,

wherein the sunscreen or daily care composition is free of ethylhexyl salicylate (also known as octisalate, also known as EHS), (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene, also known as OCR), 3,3,5-Trimethylcyclohexyl 2-hydroxybenzoate (INCI Homosalate, also known as Homomenthyl salicylate, also known as HMS), butyloctyl salicylate, and 2-hydroxy-4-methoxybenzophenone (INCI benzophenone-3, also known as oxybenzone, also known as B3), and wherein at least one of the inorganic UV filters (ii) titanium dioxide or (iii) zinc oxide is coated.

In a preferred embodiment C1 of the third aspect, the titanium dioxide has a number-average elementary particle diameter of less than 1000 nm, preferably of less than 100 nm, and in particular less than 50 nm.

In a preferred embodiment C2 of the third aspect, the zinc oxide has a number-average elementary particle diameter of less than 1000 nm, preferably of less than 200 nm, and in particular less than 150 nm.

In a preferred embodiment C3 of the third aspect, the titanium dioxide is coated, preferably wherein the titanium dioxide is a coated titanium dioxide which is a pigment that has undergone one or more surface treatments of chemical, electronic, mechanochemical and/or mechanical nature preferably with compounds selected from the group consisting of amino acids, beeswax, fatty acids, fatty alcohols, anionic surfactants, lecithins, sodium, potassium, zinc, iron or aluminium salts of fatty acids, metal alkoxides (of titanium or aluminium), polyethylene, silicones, proteins (collagen, elastin), alkanolamines, silicon oxides, metal oxides, sodium hexametaphosphate, silica and mixtures thereof, more preferably wherein the titanium oxide is coated with aluminium hydroxide, stearic acid, and mixtures thereof. In a further preferred embodiment, the titanium oxide is coated with silica, dimethicone, and mixtures thereof.

In a preferred embodiment C4 of the third aspect, the zinc oxide is uncoated or coated, preferably wherein the zinc oxide is coated with inorganic surface coatings selected from the group consisting of aluminum oxide (Al₂O₃), aluminum hydroxide Al(OH)₃, aluminum oxide hydrate, sodium hexametaphosphate (NaPO₃), sodium meta-phosphate (NaPO₃)ₙ, silicon dioxide (SiO₂), iron oxide (Fe₂O₃), and mixtures thereof; or organic surface coatings selected from the group consisting of vegetable or animal aluminum stearate, fatty acids, dimethylpolysiloxane, methylpolysiloxane, simethicone, triethoxycaprylylsilane, octyltrimethoxysilane, and mixtures thereof; or mixtures of inorganic surface coatings with organic surface coatings, more preferably triethoxycaprylylsilane.

In a preferred embodiment C5 of the third aspect, the weight ratio of the sum of titanium dioxide and zinc oxide to the bis-ethylhexyloxyphenol methoxyphenyl triazine is in the range of 1:1 to 20:1, preferably 1:1 to 12:1, and in particular 2:1 to 6:1 and/or wherein the weight ratio of the titanium dioxide to the zinc oxide is in the range of 4:1 to 1:3, preferably of 3:1 to 1:2, more preferably of 5:2 to 1:1, and in particular of 2:1 to 6:5.

In a preferred embodiment C6 of the third aspect, the sunscreen or daily care composition comprises at least one further, different UV filter, preferably wherein the weight ratio of the at least one further, different UV filter to bis-ethylhexyloxyphenol methoxyphenyl triazine (INCI bis-ethylhexyloxyphenol methoxyphenyl triazine) is more than 1:1 to 30:1.

In a preferred embodiment C7 of the third aspect, the at least one further, different UV filter is hexyl 2-[4-(diethylamino)-2-hydroxybenzoyl]benzoate (INCI diethylamino hydroxybenzoyl hexyl benzoate), preferably wherein the weight ratio of hexyl 2-[4-(diethylamino)-2-hydroxybenzoyl]benzoate (INCI diethylamino hydroxybenzoyl hexyl benzoate) to bis-ethylhexyloxyphenol methoxyphenyl triazine (INCI bis-ethylhexyloxyphenol methoxyphenyl triazine) is more than 1:1 to 30:1.

In a preferred embodiment C8 of the third aspect, the sunscreen or daily care composition does not comprise a further, different UV filter.

In a preferred embodiment C9 of the third aspect, the sunscreen or daily care composition comprises at least one compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, a fused ring cyanoacrylate derivative, polyester-25, and combinations thereof.

In a preferred embodiment C10 of the third aspect, the titanium dioxide and zinc oxide are comprised in an amount of 1 to 35 wt.-%, preferably of 5 to 25 wt.-%, and in particular of 8 to 20 wt.-%, based on the total amount of the sunscreen or daily care composition.

In a preferred embodiment C11 of the third aspect, the sunscreen or daily care composition comprises
(i) bis-ethylhexyloxyphenol methoxyphenyl triazine (INCI bis-ethylhexyloxyphenol methoxyphenyl triazine) in an amount of 1 to 10 wt.-%, preferably of 1.5 to 6 wt.-%;
(ii) titanium dioxide in an amount of 2 to 20 wt.-%, preferably of 4 to 15 wt.-%; and
(iii) zinc oxide in an amount of 1 to 20 wt.-%, preferably of 3 to 15 wt.-%;

each based on the total weight of the sunscreen or daily care composition.

In a preferred embodiment C12 of the third aspect, the sunscreen or daily care composition comprises a total of 5 to 40 wt.-% of UV filters, preferably of 9 to 30 wt.-%, and in particular of 9 to 20 wt.-%, based on the total weight of the sunscreen or daily care composition.

In a preferred embodiment C13 of the third aspect, the sunscreen or daily care composition provides a SPF of more than 15, preferably of more than 30 or a SPF of more than 50, preferably of more than 60, or a SPF of more than 90, preferably of more than 100.

In a preferred embodiment C14 of the third aspect, the sunscreen or daily care composition provides a higher SPF value than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to embodiments C to C13.

In a preferred embodiment C15 of the third aspect, the sunscreen or daily care composition provides a photostability of more than 85%, preferably of more than 90%, and in particular of more than 95%.

In a preferred embodiment C16 of the third aspect, the sunscreen or daily care composition provides a higher photostability than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to embodiments C to C15.

In a preferred embodiment C17 of the third aspect, the weight ratio of the titanium dioxide to the zinc oxide is in the range of 4:1 to 1:3, preferably of 3:1 to 1:2, more preferably of 5:2 to 1:1, and in particular of 2:1 to 6:5.

In a fourth aspect D, the present invention relates to a method of enhancing the photostability, and/or reducing the stickiness, and/or improving the water resistance, and/or improving the film homogeneity, and/or reducing fabric staining before and/or after washing, and/or reducing the gloss, and/or reducing the formulation yellowing, and/or improving the compatibility with the microbiome, preferably the human skin microbiome, of a sunscreen or daily care composition comprising titanium dioxide and zinc oxide, preferably the sunscreen or daily care composition according to aspect C and all its embodiments, by applying bis-ethylhexyloxyphenol methoxyphenyl triazine, wherein the sunscreen or daily care composition is free of ethylhexyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), 3,3,5-Trimethylcyclohexyl 2-hydroxybenzoate (INCI Homosalate), butyloctyl salicylate, and 2-hydroxy-4-methoxybenzophenone (INCI benzophenone-3) and wherein at least one of the inorganic UV filters titanium dioxide or zinc oxide is coated.

In this connection, the present invention also relates to the use of bis-ethylhexyloxyphenol methoxyphenyl triazine in a sunscreen or daily care composition comprising titanium dioxide and zinc oxide, for enhancing the photostability and/or for reducing the stickiness and/or for improving the water resistance, and/or for improving the film homogeneity, and/or reducing fabric staining before and/or after washing, and/or reducing the gloss, and/or reducing the formulation yellowing, and/or for improving the compatibility with the microbiome, preferably the humans skin microbiome, of said sunscreen or daily care composition, preferably according to aspect C and all its embodiments.

In a fifth aspect E, the present invention relates to a sunscreen of daily care composition comprising
(i) bis-ethylhexyloxyphenol methoxyphenyl triazine (INCI bis-ethylhexyloxyphenol methoxyphenyl triazine); and
(ii) titanium dioxide,

wherein the sunscreen of daily care composition is free of a further, additional UV filter.

In a preferred embodiment E1 of the fifth aspect, the sunscreen of daily care composition comprises
(i) bis-ethylhexyloxyphenol methoxyphenyl triazine (INCI bis-ethylhexyloxyphenol methoxyphenyl triazine) in an amount of 1 to 10 wt.-%, preferably of 1.5 to 6 wt.-%; and
(ii) titanium dioxide in an amount of 2 to 30 wt.-%, preferably of 4 to 15 wt.-%; each based on the total weight of the sunscreen or daily care composition.

In a sixth aspect F, the present invention relates to a sunscreen of daily care composition comprising
(i) bis-ethylhexyloxyphenol methoxyphenyl triazine (INCI bis-ethylhexyloxyphenol methoxyphenyl triazine); and
(ii) zinc oxide, wherein the sunscreen of daily care composition is free of a further, additional UV filter.

### FIGURES

FIG. 1 depicts the three absorption spectra extracted from the sunscreen simulator online tool (https://www.sunscreensimulator.basf.com/) between 290 and 320 nm relevant range for the erythema formation, corresponding to 1 wt.-% BEMT, or 1 wt.-% Benzophenone 3 (B3), or 1.75 wt.-% B3. B3 is as BEMT a lipophilic UV filter.

FIG. 2 depicts an example of the grey value (or brightness) distribution used for film homogeneity evaluation. The horizontal line in the distribution curve corresponds to the half-width-half-maximum (HWHM) used a criterion to define the applied sunscreen film homogeneity.

FIG. 3 depicts the stickiness (Force in mN) of Formulations Inv. 1, Comp. 1, and Comp. 2.

FIG. 4 depicts the stickiness (Force in mN) of Formulations Inv. 7 and Comp. 10.

FIG. 5 depicts the stickiness (Force in mN) of Formulations Inv. 10 and Comp. 13.

FIG. 6 depicts the gloss in formulations Inv. 12 and Comp. 15.

### DETAILED DESCRIPTION

Before describing in detail exemplary embodiments of the present invention, definitions which are important for understanding the present invention are given.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As used herein the term “does not comprise” or “free of” means in the context that the composition of the present invention is free of a specific compound or group of compounds, which may be combined under a collective term, that the composition does not comprise said compound or group of compounds in an amount of more than 0.8% by weight, based on the total weight of the composition. Furthermore, it is preferred that the composition according to the present invention does not comprise said compounds or group of compounds in an amount of more than 0.5% by weight, preferably the composition does not comprise said compounds or group of compounds at all.

When referring to compositions and the weight percent of the therein comprised ingredients it is to be understood that according to the present invention the overall amount of ingredients does not exceed 100% (±1% due to rounding).

The term “sunscreen composition” or “sunscreen” refers to any topical product, which absorbs and which may further reflect and scatter certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic and inorganic UV filters, as well as other ingredients or additives, e.g., emulsifiers, emollients, viscosity regulators, stabilizers, preservatives, or fragrances.

As suitable inorganic UV filters titanium dioxide, zinc oxide, and cerium oxide may be named.

The term “daily care composition” refers to any topical product, which absorbs and which may further reflect and scatter certain parts of UV radiation and is used as an everyday care product for the human body, e.g. for face or body. The daily care composition may comprise one or more active agents, e.g., organic and/or inorganic UV filters, as well as other ingredients or additives, e.g., emulsifiers, emollients, viscosity regulators, stabilizers, preservatives, or fragrances. Suitable daily care composition are according to the present invention, e.g. leave-on face and body care products.

Suitable leave-on products for face and body are, e.g. sunscreen compositions, decorative preparations, and skin care preparations.

Suitable decorative preparations are, e.g., lipsticks, nail varnishes, eye shadows, mascaras, dry and moist make-up, rouge, powders, depilatory agents and suntan lotions.

Suitable skin care preparations are e.g., moisturizing, refining, and lifting preparations. The cited daily care compositions can be in the form of creams, ointments, pastes, foams, gels, lotions, powders, make-ups, sprays, sticks or aerosols.

The term “UV filter” or “ultraviolet filter” as used herein refers to organic or inorganic compounds, which can absorb and may further reflect and scatter UV radiation caused by sunlight. UV-filter can be classified based on their UV protection curve as UV-A, UV-B, or broadband filters. In particular, organic UV filters may comprise moieties, which are based on compounds selected from the group consisting of benzophenone such as benzophenone-3 (oxybenzone), benzophenone-4, benzophenone-5, camphor such as benzylidenecamphor derivatives, anthranilic, PABA (4-aminobenzoic acid) such as PABA and ethyl PABA and ethyl dihydroxypropyl PABA and PEG-25 PABA, β, β-diphenylacrylate, cinnamic acid (especially cinnamate) such as ethylhexyl methoxycinnamate and isoamyl methoxycinnamate and DEA methoxycinnamate and isopropyl methoxycinnamate, dibenzoylmethane such as butyl methoxydibenzoylmethane and isopropyl dibenzoylmethane, benzimidazole such as phenylbenzimidazole derivatives, salicylic acid (especially salicylate) such as ethylhexyl salicylate (octisalate) and homomenthyl salicylate (homosalate) and phenyl salicylate and butyloctyl salicylate and TEA salicylate, triazine such as diethylhexyl butamido triazine and ethylhexyl triazone, aminobenzophenone, benzoxazole, and mixtures thereof. Further, the term UV filter may be understood as a substance, which is listed in the Annex VI (version of Mar. 12, 2020) of the Regulation (EC) No 1223/2009 of the European Parliament and of the Council (https://www.legislation.gov.uk/eur/2009/1223/annex/VI).

Water soluble UV filters have a solubility in water of at least 2% by weight, preferably at least 3% by weight, more preferably at least 5% by weight.

Oil soluble UV filters have a solubility in common cosmetic oils, such as C₁₂-C₁₅-alkyl benzoate, dibutyl adipate, lauryl lactate, myristyl lactate, diisopropyl sebacate, phenethyl benzoate, or dicaprylyl carbonate of at least 2% by weight, preferably at least 5% by weight, more preferably at least 7% by weight.

The term “C₁₂-C₁₅ alkyl benzoate” refers to esters of benzoic acid with fatty alcohols containing a C₁₂-C₁₅-alkyl chain. C₁₂-C₁₅ alkyl chain is defined as an alkyl chain with C₁₂, C₁₃, C₁₄ or C₁₅ chain length.

The term “emollient” relates to cosmetic specific oils used for protecting, moisturizing and lubricating the skin. The word emollient is derived from the Latin word mollire, to soften. In general, emollients prevent evaporation of water from the skin by forming an occlusive coating. They can be divided into different groups depending on their polarity index.

The term “sensitive skin” refers to skin of which the natural barrier function is weakened and has broken due to a trigger. A trigger can be for example cold weather, extremely hot water and critical ingredients, which may be included in sunscreen or daily care compositions.

The term “sun protection factor (SPF)” as used herein indicates how well the skin is protected by a sunscreen composition mainly from UV-B radiation. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimeter (mg/cm²).

The definition of “broadband” protection (also referred to as broad-spectrum or broad protection) is based on the “critical wavelength”. For broadband coverage, UV-B and UV-A protection must be provided. According to the US requirements, a critical wavelength of at least 370 nm is required for achieving broad spectrum protection. Furthermore, it is recommended by the European Commission that all sunscreen or cosmetic compositions should have an UV-A protection factor, which is at least one third of the labelled sun protection factor (SPF), e.g. if the sunscreen composition has an SPF of 30 the UVA protection factor has to be at least 10.

The term “critical wavelength” is defined as the wavelength at which the area under the UV protection curve (% protection versus wavelength) represents 90% of the total area under the curve in the UV region (290-400 nm). For example, a critical wavelength of 370 nm indicates that the protection of the sunscreen composition is not limited to the wavelengths of UV-B, i.e. wavelengths from 290-320 nm, but extends to 370 nm in such a way that 90% of the total area under the protective curve in the UV region are reached at 370 nm.

The term “administration” refers to the application of a sunscreen or daily care composition to the skin of a person.

Preferred embodiment regarding the sunscreen or daily care composition as well as the use thereof are described hereinafter. It is to be understood that the preferred embodiments of the invention are preferred alone or in combination with each other.

As indicated above, the present invention relates in one embodiment to a sunscreen or daily care composition comprising
(i) bis-ethylhexyloxyphenol methoxyphenyl triazine (INCI bis-ethylhexyloxyphenol methoxyphenyl triazine);
(ii) titanium dioxide; and
(iii) zinc oxide,
   wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), and 2,2′-Methylenbis[6-(2H-benzotriazol-2-yl)-4-(1,1,3,3-tetramethylbutyl)phenol] (bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dione (avobenzone).

In this connection, it is to be understood that consequently if the sunscreen or daily care composition further comprises 1-(4-(1,1-dimethylethyl)phenyl)-3-(4-methoxyphenyl)propane-1,3-dione (INCI butyl methoxydibenzoylmethane, also known as avobenzone) said sunscreen or daily care composition is free of (RS)-2-Ethylhexyl (2E)-3-(4-methoxyphenyl)prop-2-enoate (INCI ethylhexyl methoxycinnamate, also known as octinoxate).

In general, mixing avobenzone with octinoxate should be avoided due to the high photoinstability of this combination.

As indicated above, the present invention relates a further embodiment to a sunscreen or daily care composition comprising
(i) bis-ethylhexyloxyphenol methoxyphenyl triazine (INCI bis-ethylhexyloxyphenol methoxyphenyl triazine);
(ii) titanium dioxide; and
(iii) zinc oxide,
   wherein the sunscreen or daily care composition is free of ethylhexyl salicylate, (RS)-2-Ethylhexyl (2E)-3-(4-methoxyphenyl)prop-2-enoate (INCI ethylhexyl methoxycinnamate (octinoxate), 2-ethylhexyl 2-cyano-3,3-diphenylprop-2-enoate (INCI octocrylene), 3,3,5-Trimethylcyclohexyl 2-hydroxybenzoate (INCI Homosalate), butyloctyl salicylate, and 2-hydroxy-4-methoxybenzophenone (INCI benzophenone-3), and wherein at least one of the inorganic UV filters (ii) titanium dioxide or (iii) zinc oxide is coated.

In connection with the present invention, the following preferred embodiments regarding the sunscreen or daily care composition are relevant.

Bis-ethylhexyloxyphenol methoxyphenyl triazine (BEMT) is lipophilic and can exemplarily be provided in several forms such as a solid or in dissolved form. Tinosorb S from BASF SE or Parsol Shield from DSM are supplied as solid and need to be dissolved in the oil phase of the formulation to be efficient. To make the oil soluble BEMT filter compatible and dispersible with water, BEMT can also be encapsulated in a polymer matrix such as PMMA (e.g. market products under the trade name Tinosorb S Aqua from BASF SE), acrylates/C12-22 alkyl methacrylate copolymer (Tinosorb S Lite Aqua from BASF SE), or BEMT can be encapsulated in silica bead (e.g. market products under the trade name Sunsil-S from Sunjin).

In a preferred embodiment of the present invention, the titanium dioxide is a nano material. In this connection it is to be understood that the term “nano material” follows the recommendation of the European Commission 2011/696/EU. Accordingly, in a nano material 50% of particles, based on a number-based size distribution, are smaller than 100 nm, including constituent particles in aggregates or agglomerates. In another preferred embodiment of the present invention, the titanium dioxide is a non-nano material. Accordingly, in a non-nano material more than 50% of the particles, based on a number-based size distribution, are larger than 100 nm.

In a preferred embodiment of the present invention, the titanium dioxide has a number-average elementary particle diameter of less than 1000 nm, more preferably of less than 100 nm, and in particular less than 50 nm. It is further preferred that the titanium dioxide has a number-average elementary particle diameter of 0.1 to less than 1000 nm, more preferably of 1 to 100 nm, and in particular of 2 to 50 nm. The number-average elementary particle diameter of said titanium dioxide particles may be determined by any known in the art method such as transmission electron microscopy (TEM).

In a preferred embodiment of the present invention, the zinc oxide has a number-average elementary particle diameter of less than 1000 nm, more preferably of less than 200 nm, and in particular less than 150 nm. It is further preferred that the zinc oxide has a number-average elementary particle diameter of 0.1 to less than 1000 nm, more preferably of 1 to 200 nm, and in particular of 5 to 150 nm. The a number-average elementary particle diameter of said zinc oxide particles may be determined by any known in the art method such as transmission electron microscopy (TEM).

Preferably, the titanium dioxide has a number-average elementary particle diameter of less than 100 nm, more preferably less than 50 nm and the zinc oxide has a number-average elementary particle diameter of less than 200 nm, more preferably less than 150 nm.

In a preferred embodiment of the present invention, at least one of the inorganic UV filters (ii) titanium dioxide or (iii) zinc oxide is coated.

In a preferred embodiment of the present invention, the titanium dioxide is coated. Uncoated titanium dioxide is photocatalytic and should be avoided in sunscreens. Preferably, the titanium dioxide is a coated titanium dioxide which is a pigment that has undergone one or more surface treatments of chemical, electronic, mechanochemical and/or mechanical nature preferably with compounds selected from the group consisting of amino acids, beeswax, fatty acids, fatty alcohols, anionic surfactants, lecithins, sodium, potassium, zinc, iron or aluminium salts of fatty acids (stearic acid, metal alkoxides (of titanium or aluminium), polyethylene, silicones (such as dimethicone, cyclomethicone, polysilicone, simethicone, dimethicone/methicone copolymer and/or cyclopentasiloxane), proteins (collagen, elastin), alkanolamines, silicon oxides, metal oxides, sodium hexametaphosphate, silica and mixtures thereof, more preferably wherein the titanium oxide is coated with aluminium hydroxide, stearic acid, and mixtures thereof.

Preferably, the coated titanium dioxide pigments are titanium oxides that have been coated:
with silica, such as the product Sunveil from the company Ikeda, the product Eusolex T-AVO from the company Merck, and the product Sunsil TIN 50 from the company Sunjin;
with silica and iron oxide, such as the product Sunveil F from the company Ikeda;
with silica and alumina, such as the products Microtitanium Dioxide MT 500 SA and Microtitanium Dioxide MT 100 SA from the company Tayca and Tioveil from the company Croda;
with alumina, such as the products Tipaque TTO-55 (B) and Tipaque TTO-55 (A) from the company Ishihara and UVT 14/4 from the company Sachtleben Pigments;
with stearic acid and aluminium hydroxide, such as the products Microtitanium Dioxide MT 100 TV, MT 100 TX, MT 100 Z, MT-01 and MT 900Z from the company Tayca;
with stearic acid and alumina, such as the product Solaveil XT-300-LQ from the company Croda;
with aluminium stearate and alumina, such as the products Solaveil CT-10 W, Solaveil CT 100-LQ, Solaveil CT 200-LQ and Solaveil CT-300 LQ from the company Croda;
with silica, alumina and alginic acid, such as the product MT-100 AQ from the company Tayca;
with alumina and aluminium laurate, such as the product Microtitanium Dioxide MT 100 S from the company Tayca;
with iron oxide and iron stearate, such as the product Microtitanium Dioxide MT 100 F from the company Tayca;
with silica and alumina and treated with a silicone, such as the products Microtitanium Dioxide MT 600 SAS, Microtitanium Dioxide MT 500 SAS or Microtitanium Dioxide MT 100 SAS from the company Tayca;
with silica, alumina and aluminium stearate and treated with a silicone, such as the product STT-30-DS from the company Titan Kogyo;
with silica and a silicone, such as the product UV-Titan X 195 from the company Sachtleben Pigments, and Parsol TX from the company DSM Nutritional Products;
with silica and cetyl phosphate such as the product Eusolex T-easy from Merck;
with alumina and a silicone, such as the products Tipaque TTO-55 (S) from the company Ishihara or UV Titan M 262 (with dimethicone) from the company Sachtleben Pigments; Eusolex T-2000 (with simethicone) from Merck;
with alumina and glycerin such as the product UV-Titan M212;
with alumina and Triethoxycaprylylsilane such as the product Uv Titan M765;
with triethanolamine, such as the product STT-65-S from the company Titan Kogyo;
with stearic acid, such as the product Tipaque TTO-55 (C) from the company Ishihara;
with sodium hexametaphosphate, such as the product Microtitanium Dioxide MT 150 W from the company Tayca;
with silicone such as trimethoxycaprylylsilane or simethicone;
with octyltrimethylsilane, notably sold under the trade name T 805 by the company Degussa Silices;
with a polydimethylsiloxane, notably sold under the trade name 70250 Cardre UF TiO2SI3 by the company Cardre;
anatase/rutile TiO2 treated with a polydimethylhydrogenosiloxane, notably sold under the trade name Microtitanium Dioxide USP Grade Hydrophobie by the company Color Techniques.

Mention may also be made of titanium dioxide pigments doped with at least one transition metal such as iron, zinc or manganese and more particularly manganese. Preferably, said doped pigments are in the form of an oily dispersion. The oil present in the oily dispersion is preferably chosen from triglycerides including those of capric/caprylic acids.

In a preferred embodiment of the present invention, the zinc oxide is uncoated or coated. Preferably, the zinc oxide is coated with inorganic and/or organic surface coatings.

In a preferred embodiment of the present invention, the zinc oxide is coated with inorganic surface coating selected from the group consisting of aluminum oxide (Al₂O₃), aluminum hydroxide Al(OH)₃, aluminum oxide hydrate (also: Alumina, e.g. CAS-No.: 1333-84-2), sodium hexametaphosphate (NaPO₃), sodium meta-phosphate (NaPO₃)ₙ, silicon dioxide (SiO₂) (also: Silica, e.g. CAS-No.: 7631-86-9), iron oxide (Fe₂O₃), and mixtures thereof. It is to be understood that these inorganic surface coatings can be present on their own, in combination and/or in combination with organic surface coatings.

In a preferred embodiment of the present invention, the zinc oxide is coated with organic surface coatings selected from the group consisting of vegetable or animal aluminum stearate, fatty acids such as stearic acid or lauric acid, dimethylpolysiloxane (also: dimethicone), methylpolysiloxane (methicone), simethicone (a mixture of dimethylpolysiloxane with an average chain length of about 200 to about 350 dimethylsiloxane units and silica gel), triethoxycaprylylsilane, octyltrimethoxysilane, and mixtures thereof. It is to be understood that these organic surface coatings can be present on their own, in combination and/or in combination with inorganic surface coatings. Preferably, the zinc oxide according to the present invention is uncoated or coated with dimethicone, methicone, or triethoxycaprylylsilane, most preferably the zinc oxide according to the present invention is surface coated with triethoxycaprylylsilane.

In a preferred embodiment of the present invention, the weight ratio of the sum of titanium dioxide and zinc oxide to the bis-ethylhexyloxyphenol methoxyphenyl triazine is in the range of 1:1 to 20:1, preferably 1:1 to 12:1, and in particular 2:1 to 6:1.

In a preferred embodiment of the present invention, the weight ratio of the titanium dioxide to the zinc oxide is in the range of 4:1 to 1:3, preferably of 3:1 to 1:2, more preferably of 5:2 to 1:1, and in particular of 2:1 to 6:5.

In a preferred embodiment of the present invention, the sunscreen or daily care composition is free of 2-ethylhexyl 2-cyano-3,3-diphenylprop-2-enoate (INCI octocrylene), (RS)-2-Ethylhexyl (2E)-3-(4-methoxyphenyl)prop-2-enoate (INCI ethylhexyl methoxycinnamate), 3,3,5-Trimethylcyclohexyl 2-hydroxybenzoate (INCI Homosalate), and/or 2-hydroxy-4-methoxybenzophenone (INCI benzophenone-3).

In a preferred embodiment of the present invention, the sunscreen or daily care composition comprises at least one further, different UV filter.

Preferably, the weight ratio of the at least one further, different UV filter to bis-ethylhexyloxyphenol methoxyphenyl triazine (INCI bis-ethylhexyloxyphenol methoxyphenyl triazine) is more than 1:1 to 30:1, preferably 2:1 to 20:1, and in particular 3:1 to 10:1.

In a preferred embodiment of the present invention, the sunscreen or daily care composition comprises the at least one further, different UV filter in an amount of up to 20 wt.-%, preferably up to 15 wt.-%, more preferably up to 10 wt.-%, still more preferably up to 5 wt.-%, even more preferably up to 2 wt.-%, and in particular up to 1 wt.-%. In a preferred embodiment of the present invention, the sunscreen or daily care composition comprises the at least one further, different UV filter in an amount of 0 to 20 wt.-%, preferably 0 to 15 wt.-%, more preferably 0 to 10 wt.-%, still more preferably 0 to 5 wt.-%, even more preferably 0 to 2 wt.-%, and in particular 0 to 1 wt.-%.

The at least one further, different UV filter may be any known in the art UV filter such as an organic or inorganic UV filter.

As suitable further, different inorganic UV filter cerium oxide may be named.

As organic UV filter, UVA, UVB, and/or different broadband UV filter may be applied. In this connection it is to be understood that the at least one further, different UV filter can be present in the sunscreen or daily care composition as long as not explicitly disclaimed. Suitable UVA filters may be selected from the group consisting of disodium phenyl dibenzimidazole tetrasulfonate, hexyl 2-[4-(diethylamino)-2-hydroxybenzoyl]benzoate (INCI diethylamino hydroxybenzoyl hexyl benzoate), 1-(4-(1,1-dimethylethyl)phenyl)-3-(4-methoxyphenyl)propane-1,3-dione (INCI butyl methoxydibenzoylmethane, also known as Avobenzone), Terephthalylidene dicamphor sulfonic acid, and combinations thereof. Suitable UVB filters may be selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI Diethylhexyl Butamido Triazone), phenylbenzimidazole sulfonic acid, 2-ethylhexyl 2-cyano-3,3-diphenylprop-2-enoate (INCI octocrylene), (RS)-2-Ethylhexyl (2E)-3-(4-methoxyphenyl)prop-2-enoate (octinoxate), octisalate, and combinations thereof. Suitable different broadband UV filters may be selected from the group consisting of 2-(2H-benzotriazol-2-yl)-6-[(2-ethylhexyloxy)methyl]-4-methylphenol, 2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane), 2,2′-Methylenbis[6-(2H-benzotriazol-2-yl)-4-(1,1,3,3-tetramethylbutyl)phenol] (INCI Methylene Bis-Benzotriazolyl Tetramethylbutylphenol), Phenylene Bis-Diphenyltriazine, benzophenone-3 and combinations thereof.

In a preferred embodiment, the at least one further, different UV filter is selected from the group consisting of 2-ethylhexyl 2-cyano-3,3-diphenylprop-2-enoate (INCI octocrylene), (RS)-2-Ethylhexyl (2E)-3-(4-methoxyphenyl)prop-2-enoate (octinoxate), 1-(4-(1,1-dimethylethyl)phenyl)-3-(4-methoxyphenyl)propane-1,3-dione (Avobenzone), a combination of octocrylene and octinoxate and a combination of octocrylene and Avobenzone.

In another preferred embodiment of the present invention, the at least one further, different UV filter is selected from the group consisting of ethylhexyl triazone, 2,2′-Methylenbis[6-(2H-benzotriazol-2-yl)-4-(1,1,3,3-tetramethylbutyl)phenol] (INCI Methylene Bis-Benzotriazolyl Tetramethylbutylphenol), hexyl 2-[4-(diethylamino)-2-hydroxybenzoyl]benzoate (INCI diethylamino hydroxybenzoyl hexyl benzoate, and mixtures thereof, preferably wherein the weight ratio of the at least one further, different UV filter to bis-ethylhexyloxyphenol methoxyphenyl triazine (INCI bis-ethylhexyloxyphenol methoxyphenyl triazine) is more than 1:1 to 30:1 preferably 2:1 to 20:1, and in particular 3:1 to 10:1.

In another preferred embodiment of the present invention, the at least one further, different UV filter is hexyl 2-[4-(diethylamino)-2-hydroxybenzoyl]benzoate (INCI diethylamino hydroxybenzoyl hexyl benzoate), preferably wherein the weight ratio of hexyl 2-[4-(diethylamino)-2-hydroxybenzoyl]benzoate (INCI diethylamino hydroxybenzoyl hexyl benzoate) to bis-ethylhexyloxyphenol methoxyphenyl triazine (INCI bis-ethylhexyloxyphenol methoxyphenyl triazine) is more than 1:1 to 30:1 preferably 2:1 to 20:1, and in particular 3:1 to 10:1.

In a preferred embodiment of the present invention, if the sunscreen or daily care composition comprises hexyl 2-[4-(diethylamino)-2-hydroxybenzoyl]benzoate (INCI diethylamino hydroxybenzoyl hexyl benzoate), the weight ratio of hexyl 2-[4-(diethylamino)-2-hydroxybenzoyl]benzoate (INCI diethylamino hydroxybenzoyl hexyl benzoate) to bis-ethylhexyloxyphenol methoxyphenyl triazine (INCI bis-ethylhexyloxyphenol methoxyphenyl triazine) is more than 1:1 to 30:1 preferably 2:1 to 20:1, and in particular 3:1 to 10:1.

In a preferred embodiment of the present invention, the sunscreen or daily care composition comprises not more than 20 wt.-%, preferably not more than 15 wt.-%, more preferably not more than 10 wt.-%, still more preferably not more than 5 wt.-%, even more preferably not more than 2 wt.-%, and in particular not more than 1 wt.-% of at least one further, different UV filter, preferably as defined in the Annex VI (version of Mar. 12, 2020) of the Regulation (EC) No 1223/2009 of the European Parliament and of the Council, other than bis-ethylhexyloxyphenol methoxyphenyl triazine, titanium dioxide, and zinc oxide. In this connection it is to be understood that the term “at least one further, different UV filter” as used herein comprises one, two, or more UV filter(s), preferably as defined in the Annex VI (version of Mar. 12, 2020) of the Regulation (EC) No 1223/2009 of the European Parliament and of the Council, and which is/are different than bis-ethylhexyloxyphenol methoxyphenyl triazine, titanium dioxide, and zinc oxide. Thus, in a preferred embodiment, the sunscreen or daily care composition comprise bis-ethylhexyloxyphenol methoxyphenyl triazine, titanium dioxide, and zinc oxide but only 0 to 20 wt.-%, preferably 0 to 15 wt.-%, more preferably 0 to 10 wt.-%, still more preferably 0 to 5 wt.-%, even more preferably 0 to 2 wt.-%, and in particular 0 to 1 wt.-% of at least one further, different UV, preferably as defined in the Annex VI (version of Mar. 12, 2020) of the Regulation (EC) No 1223/2009 of the European Parliament and of the Council, other than bis-ethylhexyloxyphenol methoxyphenyl triazine, titanium dioxide, and zinc oxide.

In a preferred embodiment, the sunscreen or daily care composition comprises not more than 20 wt.-%, preferably not more than 15 wt.-%, more preferably not more than 10 wt.-%, still more preferably not more than 5 wt.-%, even more preferably not more than 2 wt.-%, and in particular not more than 1 wt.-% of UV filters derived from salicylates such as ethylhexyl salicylate, homosalate, glycol salicylate, and phenyl salicylate. In particular, the sunscreen or daily care composition is free of UV filters derived from salicylates.

In a preferred embodiment of the present invention, the sunscreen or daily care composition does not comprise a further, different UV filter (i.e. does not comprise a further, different UV filter, preferably as defined in the Annex VI (version of Mar. 12, 2020) of the Regulation (EC) No 1223/2009 of the European Parliament and of the Council, and being different than bis-ethylhexyloxyphenol methoxyphenyl triazine, titanium dioxide, and zinc oxide).

In a preferred embodiment of the present invention, the sunscreen or daily care composition is free of 1,1′-(1,4-piperazinediyl)bis[1-[2-[4-(diethylamino)-2-hydroxybenzoyl]phenyl]-methanone).

In a preferred embodiment of the present invention, the bis-ethylhexyloxyphenol methoxyphenyl triazine is added in the aqueous phase.

In another preferred embodiment of the present invention, the bis-ethylhexyloxyphenol methoxyphenyl triazine is added in the oil phase.

In a preferred embodiment of the present invention, the sunscreen or daily care composition comprises at least one compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, a fused ring cyanoacrylate derivative, polyester-25, and combinations thereof.

In a preferred embodiment of the present invention, the sunscreen or daily care composition comprises the inorganic UV filter in an amount of 1 to 35 wt.-%, preferably of 5 to 25 wt.-%, and in particular of 8 to 20 wt.-%, based on the total amount of the sunscreen or daily care composition.

Preferably, the sunscreen or daily care composition comprises titanium dioxide and zinc oxide in an amount of 1 to 35 wt.-%, preferably of 5 to 25 wt.-%, and in particular of 8 to 20 wt.-%, based on the total amount of the sunscreen or daily care composition.

In a preferred embodiment of the present invention, the sunscreen or daily care composition comprises
(i) bis-ethylhexyloxyphenol methoxyphenyl triazine (INCI bis-ethylhexyloxyphenol methoxyphenyl triazine) in an amount of 1 to 10 wt.-%, preferably of 1.5 to 6 wt.-%;
(ii) titanium dioxide in an amount of 2 to 20 wt.-%, preferably of 4 to 15 wt.-%; and
(iii) zinc oxide in an amount of 1 to 20 wt.-%, preferably of 3 to 15 wt.-%; each based on the total weight of the sunscreen or daily care composition.

In another preferred embodiment of the present invention, sunscreen or daily care composition comprises
(i) bis-ethylhexyloxyphenol methoxyphenyl triazine (INCI bis-ethylhexyloxyphenol methoxyphenyl triazine) in an amount of 1 to 8 wt.-%, preferably of 2 to 6 wt.-%;
(ii) titanium dioxide in an amount of 2 to 15 wt.-%, preferably of 4 to 10 wt.-%; and
(iii) zinc oxide in an amount of 1 to 15 wt.-%, preferably of 3 to 8 wt.-%; each based on the total weight of the sunscreen or daily care composition.

In another preferred embodiment of the present invention, sunscreen or daily care composition comprises
(i) bis-ethylhexyloxyphenol methoxyphenyl triazine (INCI bis-ethylhexyloxyphenol methoxyphenyl triazine) in an amount of 1 to 8 wt.-%, preferably of 2 to 6 wt.-%;
(ii) titanium dioxide in an amount of 5 to 20 wt.-%, preferably of more than 10 to 15 wt.-%; and
(iii) zinc oxide in an amount of 5 to 20 wt.-%, preferably of 7 to 15 wt.-%; each based on the total weight of the sunscreen or daily care composition.

In a preferred embodiment of the present invention, the sunscreen or daily care composition comprises a total of 5 to 40 wt.-% of UV filters, preferably of 8 to 35 wt.-%, more preferably of 9 to 30 wt.-% or of 9 to 35 wt.-%, still more preferably of 9 to 25 wt.-%, and in particular of 9 to 20 wt.-%, based on the total weight of the sunscreen or daily care composition.

In a preferred embodiment of the present invention, the sunscreen or daily care composition provides a SPF of more than 15, preferably of more than 30. In another preferred embodiment of the present invention, the sunscreen or daily care composition provides a SPF of more than 50, preferably of more than 60. In another preferred embodiment of the present invention, the sunscreen or daily care composition provides a SPF of more than 90, preferably of more than 100. The SPF may be determined according to any known in the art method. Preferably, the SPF is measured in vivo or in vitro. Preferably, the SPF is determined according to the “plate method” as disclosed in the examples.

In a preferred embodiment of the present invention, the sunscreen or daily care composition provides a higher SPF value than a comparison sunscreen or daily care composition comprising a different UV filter, preferably a UVA filter, a UVB filter, or a different UV broadband filter, in particular a different UV broadband filter, instead of bis-ethylhexyloxyphenol methoxyphenyl triazine, wherein the comparison sunscreen or daily care composition comprises the different UV filter, preferably the UVA filter, the UVB filter, or the different UV broadband filter, in particular the different UV broadband filter, the titanium dioxide, and the zinc oxide in equal amounts as the sunscreen or daily care composition according to the present invention. The SPF may be determined according to any known in the art method. Preferably, the SPF is measured in vivo or in vitro. Preferably, the SPF is determined according to the “plate method” as disclosed in the examples. In this connection and in connection with the following comparisons, it is to be understood that the comparison sunscreen or daily care composition may comprise a UVA filter, a UVB filter or a different UV broadband filter instead of BEMT.

In a preferred embodiment of the present invention, the sunscreen or daily care composition provides a reduced stickiness than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably different UV broadband filter, the titanium dioxide, and the zinc oxide in equal amounts as the sunscreen or daily care composition according to the present invention.

In a preferred embodiment of the present invention, the sunscreen or daily care composition provides an improved film homogeneity than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to the present invention.

In a preferred embodiment of the present invention, the sunscreen or daily care composition provides a reduced fabric staining before and/or after washing, than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to the present invention.

In a preferred embodiment of the present invention, the sunscreen or daily care composition provides an improved water resistance than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to the present invention. Preferably, the water resistance is determined according to the “solution method” as disclosed in the examples.

In a preferred embodiment of the present invention, the sunscreen or daily care composition provides a photostability of more than 85%, preferably of more than 90%, and in particular of more than 95%. Preferably, the sunscreen or daily care composition provides a photostability after 10 MED, 20 MED, and/or 50 MED, of more than 85%, preferably of more than 90%, and in particular of more than 95%. Preferably, the photostability if determined in accordance with the examples.

In a preferred embodiment of the present invention, the sunscreen or daily care composition provides a higher photostability, preferably after 50 MED, than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according the present invention. Preferably, the photostability if determined in accordance with the examples.

In a preferred embodiment of the present invention, the sunscreen or daily care composition provides a reduced formulation yellowing than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to the present invention.

In a preferred embodiment of the present invention, the sunscreen or daily care composition provides a reduced formulation yellowing than a comparison sunscreen or daily care composition comprising an additional, different UV filter, preferably a UVA, a UVB, or a different UV broadband filter, than bis-ethylhexyloxyphenol methoxyphenyl triazine, wherein the comparison sunscreen or daily care composition comprises the titanium dioxide, the zinc oxide, and the bis-ethylhexyloxyphenol methoxyphenyl triazine in equal amounts as the sunscreen or daily care composition according to the present invention.

In a preferred embodiment of the present invention, the sunscreen or daily care composition provides a reduced gloss than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to the present invention.

In a preferred embodiment of the present invention, the sunscreen or daily care composition provides a reduced gloss than a comparison sunscreen or daily care composition comprising an additional, different UV filter, preferably a UVA, a UVB, or a different UV broadband filter, than bis-ethylhexyloxyphenol methoxyphenyl triazine, wherein the comparison sunscreen or daily care composition comprises the titanium dioxide, the zinc oxide, and the bis-ethylhexyloxyphenol methoxyphenyl triazine in equal amounts as the sunscreen or daily care composition according to the present invention.

In a preferred embodiment of the present invention, the sunscreen or daily care composition provides a reduced sand adhesion than a comparison sunscreen or daily care composition comprising an additional, different UV filter (e.g. butyloctyl salicylate), preferably a UVA, a UVB, or a different UV broadband filter, than bis-ethylhexyloxyphenol methoxyphenyl triazine, wherein the comparison sunscreen or daily care composition comprises the titanium dioxide, the zinc oxide, and the bis-ethylhexyloxyphenol methoxyphenyl triazine in equal amounts as the sunscreen or daily care composition according to the present invention.

In a particularly preferred embodiment of the present invention, the sunscreen or daily care composition provides a sufficient SPF, photostability, reduced stickiness, improved water resistance, improved film homogeneity, reduced fabric staining before and/or after washing, reduced formulation yellowing, reduced gloss, and improved compatible with the microbiome, preferably the human skin microbiome.

As indicated above, the present invention relates in a second aspect to a method of enhancing the photostability, and/or reducing the stickiness, and/or improving the water resistance, and/or improving the film homogeneity, and/or reducing fabric staining before and/or after washing, and/or reducing formulation yellowing, and/or reducing gloss, and/or improving the compatibility with the microbiome, preferably the human skin microbiome of a sunscreen or daily care composition comprising titanium dioxide and zinc oxide by applying bis-ethylhexyloxyphenol methoxyphenyl triazine, wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), and 2,2′-Methylenbis[6-(2H-benzotriazol-2-yl)-4-(1,1,3,3-tetramethylbutyl)phenol] (bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dione (avobenzone).

In this connection it is to be understood that the method according to the present invention provides an enhancement of the photostability when compared to a comparison sunscreen or daily care composition comprising a different UV filter, preferably a UVA filter, a UVB filter, or a different UV broadband filter, in particular a different UV broadband filter, instead of bis-ethylhexyloxyphenol methoxyphenyl triazine, the titanium dioxide, and the zinc oxide in equal amounts as the sunscreen or daily care composition according the present invention. Equally, the stickiness, the water resistance, the fabric staining before and/or after washing, the film homogeneity, and/or the compatibility with microbiome, preferably the human skin microbiome, is evaluated when compared to a comparison sunscreen or daily care composition comprising a different UV filter, preferably a UVA filter, a UVB filter, or a different UV broadband filter, in particular a different UV broadband filter, instead of bis-ethylhexyloxyphenol methoxyphenyl triazine, the titanium dioxide, and the zinc oxide in equal amounts as the sunscreen or daily care composition according the present invention. Further, the method according to the present invention may provide a reduction of the formulation yellowing and/or reduced gloss when compared to a comparison sunscreen or daily care composition comprising a different UV filter, preferably a UVA filter, a UVB filter, or a different UV broadband filter, in particular a different UV broadband filter, instead of bis-ethylhexyloxyphenol methoxyphenyl triazine, the titanium dioxide, and the zinc oxide in equal amounts as the sunscreen or daily care composition according the present invention. In addition or alternatively, the formulation yellowing and/or the gloss can be evaluated when compared to a comparison sunscreen or daily care composition comprising the titanium dioxide, the zinc oxide, and the bis-ethylhexyloxyphenol methoxyphenyl triazine in equal amounts as the sunscreen or daily care composition according to the present invention and further comprising an additional, different UV filter, preferably a UVA, a UVB, or a different UV broadband filter, than bis-ethylhexyloxyphenol methoxyphenyl triazine.

In other words, the present invention relates to a method of enhancing the photostability of a sunscreen or daily care composition comprising titanium dioxide and zinc oxide by applying bis-ethylhexyloxyphenol methoxyphenyl triazine, wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), and 2,2′-Methylenbis[6-(2H-benzotriazol-2-yl)-4-(1,1,3,3-tetramethylbutyl)phenol] (bisoctrizole), and butyloctyl salicylate and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dione (avobenzone), wherein the photostability is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

Further, the present invention relates to a method of reducing the stickiness of a sunscreen or daily care composition comprising titanium dioxide and zinc oxide by applying bis-ethylhexyloxyhenol methoxyphenyl triazine, wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), and 2,2′-Methylenbis[6-(2H-benzotriazol-2-yl)-4-(1,1,3,3-tetramethylbutyl)phenol] (bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dione (avobenzone), wherein the stickiness is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

Further, the present invention relates to a method of improving the water resistance of a sunscreen or daily care composition comprising titanium dioxide and zinc oxide by applying bis-ethylhexyloxyphenol methoxyphenyl triazine, wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), and 2,2′-Methylenbis[6-(2H-benzotriazol-2-yl)-4-(1,1,3,3-tetramethylbutyl)phenol] (bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dione (avobenzone), wherein the water resistance is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

Further, the present invention relates to a method of improving the film homogeneity of a sunscreen or daily care composition comprising titanium dioxide and zinc oxide by applying bis-ethylhexyloxyphenol methoxyphenyl triazine, wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), and 2,2′-Methylenbis[6-(2H-benzotriazol-2-yl)-4-(1,1,3,3-tetramethylbutyl)phenol] (bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dione (avobenzone), wherein the film homogeneity is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

Further, the present invention relates to a method of reducing the fabric staining before and/or after washing (washability) of a sunscreen or daily care composition comprising titanium dioxide and zinc oxide by applying bis-ethylhexyloxyphenol methoxyphenyl triazine, wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), and 2,2′-Methylenbis[6-(2H-benzotriazol-2-yl)-4-(1,1,3,3-tetramethylbutyl)phenol] (bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dioe (avobenzone), wherein the fabric staining before and/or after washing is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

Further, the present invention relates to a method of reducing the gloss of a sunscreen or daily care composition comprising titanium dioxide and zinc oxide by applying bis-ethylhexyloxyphenol methoxyphenyl triazine, wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), and 2,2′-Methylenbis[6-(2H-benzotriazol-2-yl)-4-(1,1,3,3-tetramethylbutyl)phenol] (bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dioe (avobenzone), wherein the gloss is preferably compared to a comparison sunscreen or daily care composition comprising the titanium dioxide, the zinc oxide, and the bis-ethylhexyloxyphenol methoxyphenyl triazine in equal amounts as the sunscreen or daily care composition according to the present invention and further comprising an additional, different UV filter, preferably a UVA, a UVB, or a different UV broadband filter, than bis-ethylhexyloxyphenol methoxyphenyl triazine.

Further, the present invention relates to a method of reducing the formulation yellowing of a sunscreen or daily care composition comprising titanium dioxide and zinc oxide by applying bis-ethylhexyloxyphenol methoxyphenyl triazine, wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), and 2,2′-Methylenbis[6-(2H-benzotriazol-2-yl)-4-(1,1,3,3-tetramethylbutyl)phenol] (bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dioe (avobenzone), wherein the formulation yellowing is preferably compared to a comparison sunscreen or daily care composition comprising the titanium dioxide, the zinc oxide, and the bis-ethylhexyloxyphenol methoxyphenyl triazine in equal amounts as the sunscreen or daily care composition according to the present invention and further comprising an additional, different UV filter, preferably a UVA, a UVB, or a different UV broadband filter, than bis-ethylhexyloxyphenol methoxyphenyl triazine.

Preferably, the formulation yellowing and/or the gloss and/or sand adhesion of the inventive sunscreen or daily care composition is reduced when compared to a comparison sunscreen or daily care composition comprising UV filter such as butyloctyl salicylate.

Further, the present invention relates to a method of improving the compatibility with the microbiome of a sunscreen or daily care composition comprising titanium dioxide and zinc oxide by applying bis-ethylhexyloxyphenol methoxyphenyl triazine, wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), and 2,2′-Methylenbis[6-(2H-benzotriazol-2-yl)-4-(1,1,3,3-tetramethylbutyl)phenol] (bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dione (avobenzone), wherein the compatibility with the microbiome is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

As indicated above, the present invention relates in a fourth aspect to a method of enhancing the photostability, and/or reducing the stickiness, and/or improving the water resistance, and/or improving the film homogeneity, and/or reducing the fabric staining before and/or after washing, and/or reducing formulation yellowing, and/or reducing gloss, and/or improving the compatibility with the microbiome of a sunscreen or daily care composition comprising titanium dioxide and zinc oxide by applying bis-ethylhexyloxyphenol methoxyphenyl triazine, wherein the sunscreen or daily care composition is free of ethylhexyl salicylate, 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3) and wherein at least one of the inorganic UV filters titanium dioxide or zinc oxide is coated.

In this connection it is to be understood that the method according to the present invention provides an enhancement of the photostability when compared to a comparison sunscreen or daily care composition comprising a different UV filter, preferably a UVA, filter, a UVB filter, or a different UV broadband filter, in particular a different UV broadband filter, instead of bis-ethylhexyloxyphenol methoxyphenyl triazine, the titanium dioxide, and the zinc oxide in equal amounts as the sunscreen or daily care composition according the present invention. Equally, the stickiness, the water resistance, the fabric staining before and/or after washing, the film homogeneity, and/or the compatibility with microbiome is evaluated when compared to a comparison sunscreen or daily care composition comprising a different UV filter, preferably a UVA filter, a UVB filter, or a different UV broadband filter, in particular a different UV broadband filter, instead of bis-ethylhexyloxyphenol methoxyphenyl triazine, the titanium dioxide, and the zinc oxide in equal amounts as the sunscreen or daily care composition according the present invention. Further, the method according to the present invention may provide a reduction of the formulation yellowing and/or reduced gloss and/or sand adhesion when compared to a comparison sunscreen or daily care composition comprising a different UV filter, preferably a UVA filter, a UVB filter, or a different UV broadband filter, in particular a different UV broadband filter, instead of bis-ethylhexyloxyphenol methoxyphenyl triazine, the titanium dioxide, and the zinc oxide in equal amounts as the sunscreen or daily care composition according the present invention.

In addition, the formulation yellowing and/or the gloss and/or sand adhesion can be evaluated when compared to a comparison sunscreen or daily care composition comprising the titanium dioxide, the zinc oxide, and the bis-ethylhexyloxyphenol methoxyphenyl triazine in equal amounts as the sunscreen or daily care composition according to the present invention and further comprising an additional, different UV filter, preferably a UVA, a UVB, or a different UV broadband filter, than bis-ethylhexyloxyphenol methoxyphenyl triazine.

In other words, the present invention relates to a method of enhancing the photostability of a sunscreen or daily care composition comprising titanium dioxide and zinc oxide by applying bis-ethylhexyloxyphenol methoxyphenyl triazine, wherein the sunscreen or daily care composition is free of ethylhexyl salicylate, 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3) and wherein at least one of the inorganic UV filters titanium dioxide or zinc oxide is coated, wherein the photostability is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

Further, the present invention relates to a method of reducing the stickiness of a sunscreen or daily care composition comprising titanium dioxide and zinc oxide by applying bis-ethylhexyloxyphenol methoxyphenyl triazine, wherein the sunscreen or daily care composition is free of ethylhexyl salicylate, 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3) and wherein at least one of the inorganic UV filters titanium dioxide or zinc oxide is coated, wherein the stickiness is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

Further, the present invention relates to a method of improving the water resistance of a sunscreen or daily care composition comprising titanium dioxide and zinc oxide by applying bis-ethylhexyloxyphenol methoxyphenyl triazine, wherein the sunscreen or daily care composition is free of ethylhexyl salicylate, 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3) and wherein at least one of the inorganic UV filters titanium dioxide or zinc oxide is coated, wherein the water resistance is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

Further, the present invention relates to a method of improving the film homogeneity of a sunscreen or daily care composition comprising titanium dioxide and zinc oxide by applying bis-ethylhexyloxyphenol methoxyphenyl triazine, wherein the sunscreen or daily care composition is free of ethylhexyl salicylate, 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3) and wherein at least one of the inorganic UV filters titanium dioxide or zinc oxide is coated, wherein the film homogeneity is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

Further, the present invention relates to a method of improving the fabric staining before and/after washing of a sunscreen or daily care composition comprising titanium dioxide and zinc oxide by applying bis-ethylhexyloxyphenol methoxyphenyl triazine, wherein the sunscreen or daily care composition is free of ethylhexyl salicylate, 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3) and wherein at least one of the inorganic UV filters titanium dioxide or zinc oxide is coated, wherein the fabric staining before and/after washing is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

Further, the present invention relates to a method of improving the gloss of a sunscreen or daily care composition comprising titanium dioxide and zinc oxide by applying bis-ethylhexyloxyphenol methoxyphenyl triazine, wherein the sunscreen or daily care composition is free of ethylhexyl salicylate, 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3) and wherein at least one of the inorganic UV filters titanium dioxide or zinc oxide is coated, wherein the gloss is preferably compared to a comparison sunscreen or daily care composition comprising the titanium dioxide, the zinc oxide, and the bis-ethylhexyloxyphenol methoxyphenyl triazine in equal amounts as the sunscreen or daily care composition according to the present invention and further comprising an additional, different UV filter, preferably a UVA, a UVB, or a different UV broadband filter, than bis-ethylhexyloxyphenol methoxyphenyl triazine.

Further, the present invention relates to a method of improving the formulation yellowing of a sunscreen or daily care composition comprising titanium dioxide and zinc oxide by applying bis-ethylhexyloxyphenol methoxyphenyl triazine, wherein the sunscreen or daily care composition is free of ethylhexyl salicylate, 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3) and wherein at least one of the inorganic UV filters titanium dioxide or zinc oxide is coated, wherein the formulation yellowing is preferably compared to a comparison sunscreen or daily care composition comprising the titanium dioxide, the zinc oxide, and the bis-ethylhexyloxyphenol methoxyphenyl triazine in equal amounts as the sunscreen or daily care composition according to the present invention and further comprising an additional, different UV filter, preferably a UVA, a UVB, or a different UV broadband filter, than bis-ethylhexyloxyphenol methoxyphenyl triazine.

Preferably, the formulation yellowing and/or the gloss of the inventive sunscreen or daily care composition is reduced when compared to a comparison sunscreen or daily care composition comprising UV filter such as butyloctyl salicylate.

Further, the present invention relates to a method of improving the compatibility with the microbiome of a sunscreen or daily care composition comprising titanium dioxide and zinc oxide by applying bis-ethylhexyloxyphenol methoxyphenyl triazine, wherein the sunscreen or daily care composition is free of ethylhexyl salicylate, 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3) and wherein at least one of the inorganic UV filters titanium dioxide or zinc oxide is coated, wherein the compatibility with the microbiome is compared to a comparison sunscreen or daily care composition comprising a different UV filter, preferably a different UV broadband filter, instead of bis-ethylhexyloxyphenol methoxyphenyl triazine, the titanium dioxide, and the zinc oxide in equal amounts as the sunscreen or daily care composition according the present invention.

It is to be understood that the above embodiments of the sunscreen or daily care composition are also applicable for the second and the fourth aspect.

The present invention further relates to the use of bis-ethylhexyloxyphenol methoxyphenyl triazine in a sunscreen or daily care composition comprising titanium dioxide and zinc oxide, for enhancing the photostability, and/or for reducing the stickiness, and/or for improving the water resistance, and/or reducing the fabric staining before and/or after washing, and/or reducing the formulation yellowing, and/or reducing the gloss, and/or reducing sand adhesion, and/or for improving the film homogeneity, and/or for improving the compatibility with the microbiome, preferably the human skin microbiome of said sunscreen or daily care composition, preferably according to the present invention.

It is to be understood that the above embodiments of the sunscreen or daily care composition are also applicable for the use aspect.

As indicated above, the present invention relates in a fifth aspect to a sunscreen of daily care composition comprising
(i) bis-ethylhexyloxyphenol methoxyphenyl triazine (INCI bis-ethylhexyloxyphenol methoxyphenyl triazine); and
(ii) titanium dioxide,

wherein the sunscreen of daily care composition is free of a further, additional UV filter.

In a preferred embodiment, the sunscreen of daily care composition comprises titanium dioxide in an amount of 6.5 to 25.0 wt.-%, preferably of 6.5 to 20.0 wt.-%, more preferably of 7.0 to 14.0 wt.-%, and in particular of 7.0 to 12.0 wt.-%, based on the total weight of the sunscreen or daily care composition.

In a preferred embodiment, the sunscreen of daily care composition comprises bis-ethylhexyloxyphenol methoxyphenyl triazine (INCI bis-ethylhexyloxyphenol methoxyphenyl triazine) in an amount of 1.0 to 10.0 wt.-%, preferably of 1.0 to 7.0 wt.-%, more preferably of 1.5 to 5.0 wt.-%, and in particular of 1.5 to 2.5 wt.-%, based on the total weight of the sunscreen or daily care composition.

In a preferred embodiment of the fifth aspect of the present invention, the sunscreen of daily care composition comprises
(i) bis-ethylhexyloxyphenol methoxyphenyl triazine (INCI bis-ethylhexyloxyphenol methoxyphenyl triazine) in an amount of 1 to 10 wt.-%, preferably of 1.5 to 6 wt.-%; and
(ii) titanium dioxide in an amount of 2 to 30 wt.-%, preferably of 4 to 15 wt.-%;
   each based on the total weight of the sunscreen or daily care composition.

In a preferred embodiment of the fifth aspect of the present invention, the sunscreen of daily care composition comprises
(i) bis-ethylhexyloxyphenol methoxyphenyl triazine (INCI bis-ethylhexyloxyphenol methoxyphenyl triazine) in an amount of 1.0 to 7.0 wt.-%, preferably of 1.5 to 2.5 wt.-%; and
(ii) titanium dioxide in an amount of 6.5 to 25.0 wt.-%, preferably of 7.0 to 12.0 wt.-%;
   each based on the total weight of the sunscreen or daily care composition.

A sunscreen of daily care composition according to the fifth aspect of the present invention is in particular beneficial for the microbiome.

It is to be understood that the above embodiments of the sunscreen or daily care composition are also applicable for the fifth aspect unless the embodiments regarding zinc oxide and that the sunscreen or daily care composition may optionally comprise at least one further, different UV filter, which is not the case in the fifth aspect of the present invention.

Further, the present invention relates in a sixth aspect to a sunscreen of daily care composition comprising
(i) bis-ethylhexyloxyphenol methoxyphenyl triazine (INCI bis-ethylhexyloxyphenol methoxyphenyl triazine); and
(ii) zinc oxide,

wherein the sunscreen of daily care composition is free of a further, additional UV filter.

In a preferred embodiment of the sixth aspect of the present invention, the sunscreen of daily care composition comprises
(i) bis-ethylhexyloxyphenol methoxyphenyl triazine (INCI bis-ethylhexyloxyphenol methoxyphenyl triazine) in an amount of 1 to 10 wt.-%, preferably of 1.5 to 6 wt.-%, and in particular of 1.5 to 2.5 wt.-%; and
(ii) zinc oxide in an amount of 2 to 30 wt.-%, preferably of 4 to 15 wt.-%, and in particular of 4 to 12 wt.-%;
   each based on the total weight of the sunscreen or daily care composition.

It is to be understood that the above embodiments of the sunscreen or daily care composition are also applicable for the sixth aspect unless the embodiments regarding titanium dioxide and that the sunscreen or daily care composition may optionally comprise at least one further, different UV filter, which is not the case in the sixth aspect of the present invention.

In connection with the above embodiments, it is to be understood that the sunscreen or daily care compositions are in particularly suitable for protection of the skin from UV radiation.

Hence, the present invention further relates to the sunscreen or daily care composition as defined herein in more detail for use in protecting the human skin from UV radiation. The present invention further relates to a method of protecting the human skin from UV radiation by administration of the sunscreen or daily care composition as defined herein in more detail.

It is to be understood that the above embodiments of the sunscreen or daily care composition are also applicable for the use and the method of administering.

Further, in connection with the above embodiments, it is to be understood that the sunscreen or daily care composition may comprise at least one additive.

In one embodiment, the at least one additive is selected from the group consisting of emulsifier, emollients, viscosity regulators (thickeners), sensory enhancers, adjuvants, preservatives, perfumes and combinations thereof.

Preferred emulsifiers include
glucose derivatives such as cetearyl glucoside, arachidyl glucoside, lauryl glucoside, polyglyceryl-3 methylglucose distearate, methyl glucose sesquistearate;
sucrose derivative such as sucrose polystearate, sucrose palmitate;
sorbitol derivatives;
glycerides of fatty acids such as glyceryl stearate, glyceryl oleate;
glumatic acid derivatives such as sodium stearoyl glutamate;
sulfosuccinic acid derivatives such as disodium cetearyl sulfosuccinate;
phosphoric acid derivatives such as potassium cetyl phosphate;
fatty acid esters of polyglyceryl such as polyglyceryl-3-diisostearate, polyglyceryl-2-dipolyhydroxystearate;
oxyalkenylated organomodified silicone/polysiloxane/polyalkyl/polyether copolymers and derivatives.

Preferred emollients include
esters of linear or branched fatty acids with linear or branched fatty alcohols such as propylheptyl caprylate, coco caprylate, isopropyl myristate, ethylhexyl palmitate;
esters of aromatic carboxylic acids with linear or branched fatty alcohols such as C₁₂-C₁₅-alkyl benzoate, ethylhexyl benzoate, phenethyl benzoate;
dicarboxylic acid esters with linear or branched alcohols such as dibutyl adipate, dicaprylyl carbonate, diisopropyl sebacate;
esters of hydroxycarboxylic acids with linear or branched fatty alcohols;
esters of linear or branched fatty acids with polyhydric alcohol such as butylene glycol dicaprylate/dicaprate;
mono-, di-, tri-glycerides based on C₆-C₁₈ fatty acids such as caprylic/capric triglycerides, coco glycerides;
guerbet alcohols such as octyldodecynol;
hydrocarbons such as hydrogenated polyisobutene, mineral oil, squalene, isohexadecane;
ethers such as dicaprylyl ether;
silicone derivatives (organomodified polysiloxanes) such as dimethylpolysiloxane, cyclic silicones.

Preferred thickeners include
fatty alcohols such as cetyl alcohol, cetearyl alcohol, stearyl alcohol;
fatty acids such as stearic acid;
fatty acid esters such as myristyl stearate;
waxes such as beeswax, carnauba wax, microcrystalline wax, ceresin, ozocerite;
polysaccharides or derivatives such as xanthan gum, guar gum, agar gum, alginates, gellan gum, carraghenan;
polyacrylates or homopolymers of reticulated acrylic acids or polyacrylamides such as carbomers, acrylate copolymers, acrylate/C₁₀-C₃₀-alkyl acrylate crosspolymer, acrylate/beheneth-25 methacrylate copolymer;
silicate derivatives such as magnesium silicates;
cellulose derivatives such as hydroxypropyl cellulose.

Preferred sensory enhancers include
polyamide derivatives such as nylon-12;
polymethyl methacrylates;
silica;
mica;
polymethylsilsesquioxane;
polyethylene;
starch derivatives such as aluminum starch octenylsuccinate;
dimethicone derivatives;
boron nitride;
HDI/trimethylol hexyllactone crosspolymer.

Preferred Humectant include
1,2 pentadiol;
1,2 hexandiol;
1,2 octandiol;
1,2 decandiol;
2-Methyl-1,3-propandiol.

Preferred adjuvants include
tocopherol derivatives;
retinol derivatives;
ascorbic acid derivatives;
bisabolol;
allantoin;
panthenol;
chelating agents (EDTA, EDDS, EGTA, phytic acid, piroctone olamine);
ethylhexyl glycerin;
hydroxyacetophenone;
caprylhydroxymic acid;
propellants such as propane, butane, isobutene, dimethyl ether;
styrene/PVP or styrene acrylamide copolymers;
insect repellants such as butylacetylaminopropionate.

Preferred preservatives include
benzyl alcohol;
zingerone.

Preferred perfumes are selected from the group consisting of limonene, citral, linalool, alpha-isomethylionon, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzylalcohol, benzylbenzoate, benzylcinnamate, benzylsalicylate, bergamot oil, bitter orange oil, butylphenylmethylpropioal, cardamom oil, cedrol, cinnamal, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexylcinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin and combinations thereof.

In connection with the above preferred embodiments, it is to be understood that if the sunscreen or daily care composition comprises two or more additives, combinations of the additives as defined above are also part of the invention.

In connection with the above preferred and particularly preferred embodiments, it is to be understood that the sunscreen or daily care composition may further comprise water.

In case water is present in the sunscreen or daily care composition it is to be understood that it is preferably present in an amount of more than 5% by weight, based on the total weight of the composition. Further, it is to be understood that in case water is present in the sunscreen or daily care composition, the sunscreen or daily care composition can be an oil in water emulsion (O/W emulsion), a water in oil emulsion (W/0 emulsion), a gel cream or an oil in gel.

In one embodiment of the present invention, the sunscreen or daily care composition can be provided in different forms, e.g. gels, creams, oils, lotions, sticks, or in the form of a sprayable product.

The ingredients as above-outlined are in particular suitable for sensitive skin.

The invention further relates to the following items:
1. Sunscreen or daily care composition comprising
(i) bis-ethylhexyl methoxyphenyl triazine (INCI bis-ethylhexyloxyphenol methoxyphenyl triazine);
(ii) titanium dioxide; and
(iii) zinc oxide.
2. The sunscreen or daily care composition according to item 1, wherein the titanium dioxide has a number-average elementary particle diameter of less than 1000 nm.
3. The sunscreen or daily care composition according to item 1 or 2, wherein the zinc oxide has a number-average elementary particle diameter of less than 1000 nm.
4. The sunscreen or daily care composition according to any one of items 1 to 3, wherein the titanium dioxide is coated.
5. The sunscreen or daily care composition according to any one of items 1 to 4, wherein the zinc oxide is uncoated or coated.
6. The sunscreen or daily care composition according to any one of items 1 to 5, wherein the weight ratio of the sum of titanium dioxide and zinc oxide to the bis-ethylhexyl methoxyphenyl triazine is in the range of 1:1 to 20:1.
7. The sunscreen or daily care composition according to any one of items 1 to 6, wherein the sunscreen or daily care composition is free of 2-ethylhexyl 2-cyano-3,3-diphenylprop-2-enoate (INCI octocrylene), (RS)-2-Ethylhexyl (2E)-3-(4-methoxyphenyl)prop-2-enoate (INCI ethylhexyl methoxycinnamate), and/or 2-hydroxy-4-methoxybenzophenone (INCI benzophenone-3).
8. The sunscreen or daily care composition according to any one of items 1 to 7, wherein the sunscreen or daily care composition comprises at least one further, different UV filter.
9. The sunscreen or daily care composition according to any one of items 1 to 7, wherein the sunscreen or daily care composition does not comprise a further, different UV filter.
10. The sunscreen or daily care composition according to any one of items 1 to 9, wherein the sunscreen or daily care composition comprises at least one compound selected from the group consisting of butyloctyl salicylate, benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, a fused ring cyanoacrylate derivative, polyester-25, and combinations thereof.
11. The sunscreen or daily care composition according to any one of items 1 to 10, comprising titanium dioxide and zinc oxide in an amount of 1 to 35 wt.-%, based on the total amount of the sunscreen or daily care composition.
12. The sunscreen or daily care composition according to any one of items 1 to 11, comprising
(i) bis-ethylhexyl methoxyphenyl triazine (INCI bis-ethylhexyloxyphenol methoxyphenyl triazine) in an amount of 1 to 10 wt.-%;
(ii) titanium dioxide in an amount of 2 to 20 wt.-%; and
(iii) zinc oxide in an amount of 1 to 20 wt.-%;
each based on the total weight of the sunscreen or daily care composition.
13. The sunscreen or daily care composition according to any one of items 1 to 12, wherein the sunscreen or daily care composition comprises a total of 5 to 40 wt.-% of UV filters, based on the total weight of the sunscreen or daily care composition.
14. The sunscreen or daily care composition according to any one of items 1 to 13, providing a SPF of more than 15 or a SPF of more than 50 or a SPF of more than 90.
15. The sunscreen or daily care composition according to any one of items 1 to 14, providing a higher SPF value than a comparison sunscreen or daily care composition comprising a different UV broadband filter instead of bis-ethylhexyl methoxyphenyl triazine, wherein the comparison sunscreen or daily care composition comprises the different UV broadband filter, the titanium dioxide, and the zinc oxide in equal amounts as the sunscreen or daily care composition according to any one of item 1.
16. The sunscreen or daily care composition according to any one of items 1 to 15, providing a photostability of more than 85%.
17. The sunscreen or daily care composition according to any one of items 1 to 16, providing a higher photostability than a comparison sunscreen or daily care composition comprising a different UV broadband filter instead of bis-ethylhexyl methoxyphenyl triazine, wherein the comparison sunscreen or daily care composition comprises the different UV broadband filter, the titanium dioxide, and the zinc oxide in equal amounts as the sunscreen or daily care composition according to item 1.
18. Method of enhancing the photostability, and/or reducing the stickiness, and/or improving the water resistance, and/or improving the film homogeneity, and/or improving the compatibility with the microbiome of a sunscreen or daily care composition comprising titanium dioxide and zinc oxide by applying bis-ethylhexyl methoxyphenyl triazine.
19. A sunscreen of daily care composition comprising (i) bis-ethylhexyl methoxyphenyl triazine (INCI bis-ethylhexyloxyphenol methoxyphenyl triazine); and
(ii) titanium dioxide, wherein the sunscreen of daily care composition is free of a further, additional UV filter.
20. The sunscreen or daily care composition according to item 19, comprising
(i) bis-ethylhexyl methoxyphenyl triazine (INCI bis-ethylhexyloxyphenol methoxyphenyl triazine) in an amount of 1 to 10 wt.-%; and
(ii) titanium dioxide in an amount of 2 to 30 wt.-%;
each based on the total weight of the sunscreen or daily care composition.

The present invention is further illustrated by the following examples.

### EXAMPLES

### Methods

### SPF In Vitro (Plate Method)

SPF in vitro is based on the assessment of UV-transmittance through a thin film of sunscreen sample spread on roughened substrate—SB6 PMMA-plates (4-5 μm roughness, area 4.8×4.8 cm) from Helioscreen, Paris. The sunscreen formulation to be tested was applied on the PMMA plate with an amount of 1.2 mg/cm². Three plates and 5 measurements per plate were done per formulation. The formulation was distributed manually as equal as possible on the plate. The plates applied with formulation were stored 30 minutes at room temperature and in a dark place prior the measurement.

In vitro transmission measurements were performed from 290-400 nm with 1 nm steps with the Labsphere UV Transmittance Analyzer UV 2000S. the SPF in vitro values were calculated with the formula:

$$SPF = \frac{{\sum }_{290 nm}^{400 nm} {s}_{er} ( λ ) , S ( λ )}{{\sum }_{290 nm}^{400 nm} {s}_{er} ( λ ) , S ( λ ) , T ( λ )}$$

with:
sₑᵣ(λ)=erythema action spectrum
S(λ)=spectral irradiance
T(λ)=measured light transmittance.

Further, UVA-PF in vitro was determined based on ISO 24443, which is based on the assessment of UV-transmittance through a thin film of sunscreen sample spread on roughened substrate—SB6 PMMA-plates (4-5 μm roughness, area 4.8×4.8 cm) from Helioscreen, Paris. The sunscreen formulation to be tested was applied on the PMMA plate with an amount of 1.2 mg/cm². Three plates and 5 measurements per plate were done per formulation. The formulation was distributed manually as equal as possible on the plate.

The plates applied with formulation were stored 30 minutes at room temperature and in a dark place prior the measurement.

### Measurements:

### Step 1

In vitro transmission measurement were performed from 290-400 nm with 1 nm steps prior to any UV irradiation with the Labsphere UV Transmittance Analyzer UV 2000S. The initial UV transmission spectrum was acquired and the SPF in vitro values were calculated before irradiation (mean SPF of all 15 values (from the three plates and 5 measurements per plate))

### Step 2

A mathematical adjustment of the initial UV spectrum was performed with a coefficient “c” as described in ISO 24443 and the UVA-PF in vitro was calculated before irradiation.

### Step 3

The UV irradiation with Atlas Suntest CPS+ Solar simulator of the formulation was calculated with the UV Dose D as described in ISO 24443.

### Step 4

In vitro transmission measurement after UV exposure and acquisition of the UV transmission spectrum after irradiation were performed. The in vitro SPF value after irradiation was determined. (average of 15 values from the three plates and 5 measurements per plate)

### Step 5

A mathematical adjustment of the UV spectrum after irradiation was performed with the same coefficient “c” (determined step 2). The UVA-PF in vitro was calculated after UV exposure.

### SPF (In Vivo)

The SPF in vivo was measured according to the norm ISO24444:2019 which uses the erythemal response of the skin to ultraviolet. The SPF in vivo is the ratio calculated from the energies required to induce a minimum erythemal response with and without tested sunscreen product applied to the skin of human test subjects.

### Whitening Delta L

Whitening is based on the measurement of the L parameter from the CIELAB color space or Lab system which expresses the perceptual lightness which defines black at a value of 0 and white at a value of 100.

The sunscreen formulation to be tested is applied on SB6 PMMA-plates (4-5 μm roughness, area 4.8×4.8 cm) with an amount of 1.2 mg/cm². Three plates and 4 measurements per plate were done per formulation. The formulation was distributed manually as equal as possible on the plate. The plates applied with formulation were stored 30 minutes at room temperature and in a dark place prior the measurement. The Lightness L of blank plate with glycerin and of the sample plate with the tested formulation was measured with a remission spectrometer (Datacolor SF400, light D65, observer 10°).

ΔL reflects the changes of the dark-light parameter and is calculated as the difference between the sample plate and the blank plate each on a black background. The higher ΔL, the higher is the lightness and thus the whitening effect, the lower ΔL, the more transparent (the less whitening) is the sample.

### Stickiness

A collagen biomatrix film membrane served as skin substitute, its wall thickness is about 146 μm and it has a roughness Ra comparable to human skin. 2 mg/cm² of tested sunscreen was applied on the collagen biomatrix film membrane using a brush with synthetic bristles on a surface area on 5×6 cm. The collagen membrane substrate was fixed on a sample table maintained at 36° C. using double sided adhesive tape.

The sample table was located under a texture analyzer (TA.XT plusC) which measured the force (in mN) required to lift the silicone specimen (resembling a human finger) off the surface of the sunscreen-covered skin substitute.

The force was measured 20 times every 10 seconds. Five measurements per formulation were done. The measured force is associated to the stickiness; the more sticky a formulation, higher is the measured force needed to remove the silicone specimen from the surface of the applied sunscreen; inversely, the lower the measured force, the less sticky is a formulation.

The first value at t=0 s corresponds to the measurement of control of the untreated collagen membrane which is set to 100.

### Film Homogeneity

2 μl/cm² of tested sunscreen was applied on forearms of volunteers for 10 s on a skin area which was about 5×5 cm and manually spread with the finger. Each formulation was tested on 12 volunteers.

Cross-polarized UV-image of the sunscreen-covered skin area were taken using a UV sensitive camera with a peak at 360 nm in combination with a modified flashgun which emits UV light at low intensities.

A circular section of the obtained picture with a diameter of 4 cm was cropped within the middle of the application area for further evaluation. Each circular image was then processed to determine the grey value distribution indicating the spatial UV light reflection in form of an histogram. The resulting bell-shaped curve of the distribution provides information of the homogeneity of the applied sunscreen film. Indeed the narrower the distribution, the more homogeneous is the applied sunscreen film. To get a value which represents the film homogeneity the half-width-half-maximum value (HWHM) of each distribution curve was calculated (cf. FIG. 2). The lower the HWHM, the more homogeneous is the applied sunscreen film.

### Antibacterial Activity to Evaluate the Microbial Compatibility

The antibacterial activity to evaluate the microbial compatibility was tested using a suspension of Staphylococcus epidermidis (DSM 1798) because it is a typical representative of the human skin flora.

Each test sample is contaminated at t=0 h with a bacterial solution of S. epidermidis in sodium chloride and homogenized. Samples are stored at 36° C. for the test duration of 24 h.

At various time points sampling and microbial enumeration are done. At the sampling intervals 100 μl from each sample are spread out on the surface of tryptone soy agar plates containing neutralizer. Additionally, a 1:10 dilution is done from each sample in neutralizer and inactivated for 15-30 minutes followed by a further 1:10 dilution in saline. Aliquots from both dilutions are spread out on the surface of tryptone soy agar plates without neutralizer, too. The same procedure was done for a control containing S. epidermidis in sodium chloride to verify that the bacterial growth conditions in the test are fine.

After incubation of the agar plates at 36° C. for 2 days the colony forming units (cfu) on each countable agar plate are counted to determine the total number of cfu/ml by considering dilution steps and sample amount spread on the agar surface.

The 0 h value of each sample is only determined by calculation considering the cell counts of the overnight culture of S. epidermidis and the dilution factor used for inoculation of the samples.

### Water Resistance In Vitro Solution Method

The in vitro water resistance was determined via the “solution method” according to Sohn et al. “In vitro water resistance testing using SPF simulation based in spectroscopic analysis of rinsed sunscreen” Int J Cosmet Sci (2018) 1-9 using M14 EMA plates.

The M14 EMA plates are based on ethylene methacrylate acid copolymers having the dimension of 5 cm×5 cm. These conditions are understood to be the current most sufficient approach for determining the in vitro water resistance of formulations.

On each plate an amount of 2 mg/cm² (corresponding to 50 mg of the formulation per plate) was applied. In total, four plates were prepared per sunscreen. After sunscreen application followed by an equilibration period of 15 min, two of the four plates were immersed in a water bath. The two remaining plates were kept at ambient temperature in dark.

### Water Immersion Condition

The sunscreen-covered plates were attached using a system of strips from Tesa Hook & Loop tape at a defined height on the edges of a 1000-mL beaker (diameter of 10 cm). The beaker was filled with 500 mL of distilled water with controlled temperature of 30° C.±2° C. The water was stirred with a speed of 300 rpm for 20 min using a three-bladed propeller stirrer in a equidistant position from the attached plates. Afterwards, the plates were removed from the emptied beaker and were allowed to dry at ambient temperature for 30 min before continuation.

Spectroscopic measurements and determination of the water resistance value After immersion, each of the four plates (two subjected and two not subjected to water immersion) was put in a separate beaker and washed using 20 mL of a solvent mixture composed of THF/ethanol/neutrol TE (50:48:2). To collect the sunscreen from the plate, each plate was rinsed 10 times using the same solvent with a Pasteur pipette holding the plate out of the solvent with tweezers to remove visually the entire sunscreen residue from the substrate plate. The solvent/formulation solution was diluted (1:40 dilution) and filled into a 1-cm quartz cuvette for UV spectroscopic measurements from 290 to 400 nm in 5 nm step using a Lambda 20 device. The result is delivered in form of absorbance data, Aᵣₛ(λ), used for the calculation of the corresponding in silico SPF value by means of a computational method developed to this purpose. The absorbance data corresponding to the two plates not subjected to water immersion delivered the static in silico SPF, whereas the data of two plates subjected to water immersion delivered the wet in silico SPF (corresponding to the value “SPF in vitro after water immersion”). The water resistance retention was deduced from UV transmittance measurement of the respective sunscreen solution resulting from the washing of the substrate plate with and without water immersion.

As different plates are used to determine the static in silico and the wet in silico SPF, the % WRR is calculated from the ratio between the average wet and average static in silico SPF as described in the equation below:

$$\% {WRR}_{( solution method )} = \frac{( Wet in silico SPFaverage - 1 )}{( Static in silico SPFaverage - 1 )} \text{?} 100$$

$$\text{?} \text{indicates text missing or illegible when filed}$$

### Water Resistance (In Vivo, 40 Min)

The SPF in vivo was measured according to the norm ISO1627:2020 relative to the water immersion procedure and ISO18861:2020 relative to the percentage of water resistance.

The percentage of water resistance is calculated form the SPF in vivo measured for a non-water immersed condition (static SPF) and a water immersed condition (SPF post water immersion). The SPF in vivo used for the determination of the water resistance was measured according to the norm ISO24444:2019.

For a 40 min water resistance test, the Product treated skin is immersed in water according to the process described in ISO 16217:2020, the following sequence of immersion and rest period is followed:
20 min of immersion of the test subjects with water circulated for the full period;
5 min to 20 min drying time with no toweling permitted between immersion periods.

For 40 min water resistance, this sequence is repeated two times

An individual percentage of water resistance (% WRi) value is then calculated for each individual subject according to Formula:

$$\% WR SPF / SPF i iwr is = ( - 1 ) ( - 1 ) \times 100 \qquad ( 1 )$$

Where, SPF is the individual SPF before water immersion; MEDisp/MEDisu; SPFiwr is the individual SPF post water immersion; MEDiwrp/MEDiwru. Both measured on the same subject.

The mean percentage of water resistance (% WR) for the product is then expressed as the arithmetic mean of the “n” individual % WR values (% WRi)

Fabric Staining Before and/or after Washing Delta b*

The fabric staining is based on the measurement of the b parameter from the Commission Internationale de l'Eclairage (CIE) LAB color space. The color difference between a formulation stain on a textile and the textile alone is determined to determine fabric staining of the formulation. Similarly, the color difference can be evaluated after washing to determine the effect of washing on a potential reduction of fabric staining, i.e. the washability. The color difference is indicated in absolute color coordinates and can be referred to as Delta Δ. First, the colors of the two samples are measured. Then, the difference can be calculated using the resulting colorimetric values, the CIE L*a*b coordinates; L* indicates lightness, a* is the red/green coordinate, and b* is the yellow/blue coordinate. The total color difference between two samples is expressed by ΔE*:

$$Δ E *= [ Δ L * 2 + Δ a * 2 + Δ b * 2 ] 1 / 2$$

As the color of a sunscreen composition stain is typically yellow, the visibility of the sunscreen composition stain is expressed by Δb*, ΔE* is therefore for the purpose of the present invention considered to be identical to Δb*. In order to determine fabric staining due to the sunscreen formulation according to the present invention, we used the Δb* value; Δb* (b* sample 1−b* sample 2) is the difference in yellow and blue (+ is yellower, − is bluer). The lower the color difference, the lower is the fabric staining. Further, Δb* value were measured after 1 washing in order to determine the washability. The lower the color difference after washing in comparison to the value before washing, the more has fabric staining been reduced, i.e. the better the washability of the sunscreen composition from textiles.

The measurement was performed with a spectrophotometer using the following instrument: Remission Spectrometer Datacolor SF 400 (light D65, observer 10°). The sunscreen composition was diluted in water at a concentration of 10% by weight based on the total weight of the diluted mixture. 300 mg (15 mg/cm²) of the diluted mixture was applied on a white textile (whiteness adjusted to: WG 170±10, after washing WG 190±10). For determining the washability, a washing cycle was performed in a washing machine using a temperature of 40° C. for 20 minutes and Persil Universal Gel obtained from Henkel (3 g on stain). Each textile was washed separately.

### Color (Yellowing) of the Formulation

The evaluation of the color of the bulk formulation and the corresponding yellow tone is based on the measurement of the b parameter from the CIELAB color space or Lab system. The b parameter expresses the yellow/blue coordinate (+ is yellower, − is bluer). The measurement was performed with a spectrophotometer using the following instrument: remission Spectrometer Datacolor SF400 (light D65, observer 100). The formulation was filled uniformly without air bubbles into a transparent petri dish (diameter size 58 mm, height 15 mm). Four measurements were performed on different places of the petri dish surface.

### Gloss

A collagen biomatrix (CBM) film membrane served as skin substitute, its wall thickness is about 146 μm and it has a roughness Ra comparable to human skin. The CBM is cut into a 5×5 cm sheet and adhered to the center of a plastic card using double sided adhesive tape. The adhered CBM is then wiped-off with ethanol soaked cosmetic paper tissues. 2 mg/cm² of tested formulation was applied on the collagen biomatrix film membrane using a brush with synthetic bristles on the surface area of 5×5 cm. and the card is positioned below the digital camera (“Canon S12” (ISO-80, f-number: F/1.8, shutter speed: 1/60 sec) with a curvature of 0.75-0.85. A round LED panel with a linear polarization filter and emitting warm white light is mounted approximately 40 cm above the measurement area. The digital camera is fixed on a tripod approximately 15 cm above the measurement area. A 90°-switchable linear polarization filter is mounted in front of the lens. This setup provides the means to take cross and parallel polarized images. The cross polarized image contains the information of the color of the probe and the parallel polarized image contains the information of the color plus gloss of the same probe. The image data can be specifically subtracted from one another to obtain the image containing only gloss of the tested probe. The resulting subtracted image is then evaluated using the image processing software Image J, which provides the grey values distribution which relates to the gloss intensity of the probe (the higher the mean grey value, the higher the gloss intensity). The gloss intensity of the collagen biomatrix (CBM) without the applied tested formulation is set to a value of 0 as reference point. The gloss intensity of tested formulation is given as a change to this reference point (CBM without formulation).

### Formulations

TABLE 1
Water/Oil Formulations high SPF (expected; https://www.sunscreensimulator.basf.com/);
Form. denotes Formulation

| | | Form. | Form. | Form. | Form. | Form. |
|---|---|---|---|---|---|---|
| | INCI | Inv. 1 | Inv. 4 | Comp. 1 | Comp. 2 | Comp. 3 |
| A | Polyglyceryl-2 | 4.00 | 4.00 | 4.00 | 4.00 | 4.00 |
| | Dipolyhydroxystearate | | | | | |
| | Polyglyceryl-3 | 3.00 | 3.00 | 3.00 | 3.00 | 3.00 |
| | Diisostearate | | | | | |
| | C12-15 Alkyl | 8.00 | 8.00 | 8.00 | 8.00 | 8.00 |
| | Benzoate | | | | | |
| | Dicaprylyl carbonate | 8.00 | 8.00 | 8.00 | 8.00 | 8.00 |
| | Dibutyl Adipate | 10.00 | 10.00 | 10.00 | 10.00 | 10.00 |
| | Hydrogenated Castor | 1.00 | 1.00 | 1.00 | 1.00 | 1.00 |
| | Oil | | | | | |
| | Dicaprylyl Carbonate, | 5.00 | 5.00 | 5.00 | 5.00 | 5.00 |
| | Stearalkonium | | | | | |
| | Hectorite, Propylene | | | | | |
| | Carbonate | | | | | |
| | Phenoxyethanol, | 0.80 | 0.80 | 0.80 | 0.80 | 0.80 |
| | Ethylhexylglycerin | | | | | |
| | Zinc Oxide (nano), | 5.00 | | 5.00 | 10.00 | 5.00 |
| | Triethoxycaprylylsilane | | | | | |
| | Titanium Dioxide | 8.20 | 6.80 | 8.20 | 11.60 | 8.20 |
| | (nano), Aluminum | | | | | |
| | Hydroxide, Stearic | | | | | |
| | Acid | | | | | |
| | Bis- | 4.00 | 4.00 | | | |
| | Ethylhexyloxyphenol | | | | | |
| | Methoxyphenyl | | | | | |
| | Triazine | | | | | |
| | Benzophenone-3 | | | 4.00 | | 7.00 |
| B | Aqua | qsp 100% | qsp 100% | qsp 100% | qsp 100% | qsp 100% |
| | Glycerin | 5.00 | 5.00 | 5.00 | 5.00 | 5.00 |
| | Xanthan Gum | 0.30 | 0.30 | 0.30 | 0.30 | 0.30 |

Formulations Inv. 1, Inv. 4, and Comp. 1 to Comp. 3 were manufactured as follows: Part A was heated to 80° C. under stirring without TiO2 and ZnO. When part A was homogenous TiO2 and ZnO (if present) were added, homogenised with ultra turrax device till TiO2 and ZnO (if present) were well dispersed and the part was homogeneous.

Part B was heated to 80° C. under stirring and added into part A under stirring, followed by homogenising with ultra turrax device.

The formulation was cooled down to room temperature under stirring.

TABLE 2a
Oil/Water Formulations medium SPF (expected;
https://www.sunscreensimulator.basf.com/)

| | | Formulation | Formulation | Formulation | Formulation |
|---|---|---|---|---|---|
| | INCI | Inv. 2 | Comp. 4 | Comp. 5 | Comp. 6 |
| A | Sucrose Polystearate, | 3.00 | 3.00 | 3.00 | 3.00 |
| | Cetyl Palmitate | | | | |
| | Disodium Cetearyl | 1.00 | 1.00 | 1.00 | 1.00 |
| | Sulfosuccinate | | | | |
| | Cetearyl Alcohol | 2.00 | 2.00 | 2.00 | 2.00 |
| | Propylheptyl caprylate | 5.00 | 5.00 | 5.00 | 5.00 |
| | C12-15 Alkyl Benzoate | 8.00 | 8.00 | 8.00 | 8.00 |
| | Dibutyl Adipate | 10.00 | 10.00 | 10.00 | 10.00 |
| | Phenoxyethanol and | 1.00 | 1.00 | 1.00 | 1.00 |
| | Ethylhexylglycerin | | | | |
| | (Euxyl PE 9010) | | | | |
| | Zinc Oxide (nano), | 3.00 | 3.00 | 10.00 | 3.00 |
| | Triethoxycaprylylsilane | | | | |
| | Titanium Dioxide | 5.50 | 5.50 | 6.80 | 5.50 |
| | (nano), Aluminum | | | | |
| | Hydroxide, Stearic Acid | | | | |
| | Bis-Ethylhexyloxyphenol | 2.50 | | | |
| | Methoxyphenyl | | | | |
| | Triazine | | | | |
| | Benzophenone-3 | | 4.50 | | 2.50 |
| B | Aqua | qsp 100% | qsp 100% | qsp 100% | qsp 100% |
| | Glycerin | 2.00 | 2.00 | 2.00 | 2.00 |
| | Xanthan Gum | 0.20 | 0.20 | 0.20 | 0.20 |
| | Sodium EDTA | 0.20 | 0.20 | 0.20 | 0.20 |

TABLE 2b
Oil/Water Formulations medium SPF (expected;
https://www.sunscreensimulator.basf.com/);
Form. denotes Formulation

| | | Form. | Form. |
|---|---|---|---|
| | INCI | Inv. 5 | Inv. 6 |
| A | Sucrose Polystearate, Cetyl Palmitate | 3.00 | 3.00 |
| | Disodium Cetearyl Sulfosuccinate | 1.00 | 1.00 |
| | Cetearyl Alcohol | 2.00 | 2.00 |
| | Propylheptyl caprylate | 5.00 | 5.00 |
| | C12-15 Alkyl Benzoate | 8.00 | 8.00 |
| | Dibutyl Adipate | 10.00 | 10.00 |
| | Phenoxyethanol and | 1.00 | 1.00 |
| | Ethylhexylglycerin (Euxyl PE 9010) | | |
| | Zinc Oxide (nano), | | 3.00 |
| | Triethoxycaprylylsilane | | |
| | Titanium Dioxide (nano), Aluminum | 6.80 | 5.50 |
| | Hydroxide, Stearic Acid | | |
| | Bis-Ethylhexyloxyphenol | 2.50 | |
| | Methoxyphenyl Triazine | | |
| B | Aqua | qsp 100% | qsp 100% |
| | Glycerin | 2.00 | 2.00 |
| | Xanthan Gum | 0.20 | 0.20 |
| | Sodium EDTA | 0.20 | 0.20 |
| C | Aqua, Bis-Ethylhexyloxyphenol | | 12.50 |
| | Methoxyphenyl Triazine, Acrylates/ | | |
| | C12-22 Alkyl Methacrylate Copolymer * | | |

* trade name Tinosorb S Lite Aqua, 12.5% of Tinosorb S Lite Aqua corresponds to 2.5% active of Bis-Ethylhexyloxyphenol Methoxyphenyl Triazine

TABLE 2c
Oil/Water Formulations high SPF (expected; https://www.sunscreensimulator.basf.com/);
Form. denotes Formulation

| | | Form. | Form. | Form. | Form. | Form. |
|---|---|---|---|---|---|---|
| | INCI | Inv. 7 | Comp. 9 | Comp. 10 | Comp. 11 | Comp. 12 |
| A | Sucrose Polystearate, Cetyl | 3.00 | 3.00 | 3.00 | 3.00 | 3.00 |
| | Palmitate | | | | | |
| | Disodium Cetearyl | 1.00 | 1.00 | 1.00 | 1.00 | 1.00 |
| | Sulfosuccinate | | | | | |
| | Cetearyl Alcohol | 2.00 | 2.00 | 2.00 | 2.00 | 2.00 |
| | Propylheptyl caprylate | 8.00 | 8.00 | 8.00 | 8.00 | 8.00 |
| | C12-15 Alkyl Benzoate | 8.00 | 8.00 | 8.00 | 8.00 | 8.00 |
| | Dibutyl Adipate | 10.00 | 10.00 | 10.00 | 10.00 | 10.00 |
| | Phenoxyethanol and | 1.00 | 1.00 | 1.00 | 1.00 | 1.00 |
| | Ethylhexylglycerin (Euxyl | | | | | |
| | PE 9010) | | | | | |
| | Zinc Oxide (nano), | 5.00 | 10.00 | 5.00 | 5.00 | 5.00 |
| | Triethoxycaprylylsilane | | | | | |
| | Titanium Dioxide (nano), | 8.20 | 11.60 | 8.20 | 8.20 | 8.20 |
| | Aluminum Hydroxide, | | | | | |
| | Stearic Acid | | | | | |
| | Bis-Ethylhexyloxyphenol | 4.00 | | | | |
| | Methoxyphenyl Triazine | | | | | |
| | Octyl Triazone | | | 4.00 | | |
| | Octocrylene | | | | 4.00 | |
| | Diethylamino | | | | | 4.00 |
| | hydroxybenzoyl hexyl | | | | | |
| | benzoate | | | | | |
| B | Aqua | qsp 100% | qsp 100% | qsp 100% | qsp 100% | qsp 100% |
| | Glycerin | 2.00 | 2.00 | 2.00 | 2.00 | 2.00 |
| | Xanthan Gum | 0.20 | 0.20 | 0.20 | 0.20 | 0.20 |
| | Sodium EDTA | 0.20 | 0.20 | 0.20 | 0.20 | 0.20 |

Formulations Inv. 2, Comp. 4 to Comp. 6, Inv. 5 to Inv. 7, Comp. 9, Comp. 10, Comp. 11, and Comp. 12 were manufactured as follows: Part A was heated to 80° C. under stirring without TiO2 and ZnO. When part A was homogenous TiO2 and ZnO (if present) were added, homogenised with ultra turrax device till TiO2 and ZnO (if present) were well dispersed and the part was homogeneous.

Part B was heated to 80° C. under stirring and part A was added into part B under stirring, followed by homogenising with ultra turrax device.

The formulation was cooled down to room temperature under stirring. If present, part C was added subsequently.

TABLE 2d
Oil/Water Further Formulations; Form. Denotes Formulation.

| | | Form. | Form. | Form. | Form. | Form. | Form. |
|---|---|---|---|---|---|---|---|
| | INCI | Inv. 10 | Comp. 13 | Inv. 11 | Comp. 14 | Inv. 12 | Comp. 15 |
| A1 | Sucrose Polystearate, | 3.00 | 3.00 | | | 3.00 | 3.00 |
| | Cetyl Palmitate | | | | | | |
| | Disodium Cetearyl | 1.00 | 1.00 | | | 1.00 | 1.00 |
| | Sulfosuccinate | | | | | | |
| | Lauryl Glucoside (and) | | | 3.00 | 3.00 | | |
| | Polyglyceryl-2 | | | | | | |
| | Dipolyhydroxystearate | | | | | | |
| | (and) Glycerin | | | | | | |
| | Beheneth-25 | | | 1.00 | 1.00 | | |
| | Polyhydroxystearic Acid | | | 1.00 | 1.00 | | |
| | Cetearyl Alcohol | 2.00 | 2.00 | | | 2.00 | 2.00 |
| | Propylheptyl caprylate | 10.00 | 5.00 | 10.00 | 5.00 | 11.00 | 8.00 |
| | C12-15 Alkyl Benzoate | | | | | 8.00 | 8.00 |
| | Dibutyl Adipate | | | | | 10.00 | 10.00 |
| | Phenoxyethanol and | 1.00 | 1.00 | 1.00 | 1.00 | 1.00 | 1.00 |
| | Ethylhexylglycerin (Euxyl | | | | | | |
| | PE 9010) | | | | | | |
| | Zinc Oxide (nano), | 3.00 | 3.00 | 3.00 | 3.00 | 5.00 | 5.00 |
| | Triethoxycaprylylsilane | | | | | | |
| | Titanium Dioxide (nano), | 5.50 | 5.50 | 5.50 | 5.50 | 8.20 | 8.20 |
| | Aluminum Hydroxide, | | | | | | |
| | Stearic Acid | | | | | | |
| | Bis-Ethylhexyloxyphenol | 2.50 | 2.50 | 2.50 | 2.50 | 4.00 | 4.00 |
| | Methoxyphenyl Triazine | | | | | | |
| | Butyloctyl Salicylate | | 5.00 | | 5.00 | | 3.00 |
| A2 | Hydroxyethyl | | | 2.00 | 2.00 | | |
| | Acrylate/Sodium | | | | | | |
| | Acryloyldimethyl Taurate | | | | | | |
| | Copolymer | | | | | | |
| B | Aqua | qsp | qsp | qsp | qsp | qsp | qsp |
| | | 100% | 100% | 100% | 100% | 100% | 100% |
| | Glycerin | 2.00 | 2.00 | 2.00 | 2.00 | 2.00 | 2.00 |
| | Xanthan Gum | 0.20 | 0.20 | 0.20 | 0.20 | 0.20 | 0.20 |
| | Sodium EDTA | 0.20 | 0.20 | 0.20 | 0.20 | 0.20 | 0.20 |

Formulations Inv. 10, Inv. 11, Inv. 12, Comp. 13, Comp. 14 and Comp. 15 were manufactured as follows: Part A1 was heated to 80° C. under stirring without TiO2 and ZnO. When part A1 was homogenous TiO2 and ZnO were added, part A1 was homogenised with ultra turrax device till TiO2 and ZnO were well dispersed and the part was homogeneous. Then part A2 (if present) was added into A1. Part B was heated to 80° C. under stirring and part A1(/A2 if present) was added to part B under stirring, followed by homogenizing with ultra turrax device. The formulation was cooled down to room temperature under stirring.

TABLE 2e
Oil/Water Further Formulations; Form. denotes Formulation.

| | | Form. | Form. |
|---|---|---|---|
| | INCI | Inv. 8 | Inv. 9 |
| A | Sucrose Polystearate, Cetyl Palmitate | 3.00 | 3.00 |
| | Disodium Cetearyl Sulfosuccinate | 1.00 | 1.00 |
| | Cetearyl Alcohol | 2.00 | 2.00 |
| | Propylheptyl caprylate | 8.00 | 8.00 |
| | C12-15 Alkyl Benzoate | 8.00 | 8.00 |
| | Dibutyl Adipate | 10.00 | 10.00 |
| | Phenoxyethanol and Ethylhexylglycerin | 1.00 | 1.00 |
| | (Euxyl PE 9010) | | |
| | Diethylhexyl 2,6-Naphthalate | 3.00 | |
| | Polyester-8 | | 3.00 |
| | Zinc Oxide (nano), Triethoxycaprylylsilane | 5.00 | 5.00 |
| | Titanium Dioxide (nano), Aluminum | 8.20 | 8.20 |
| | Hydroxide, Stearic Acid | | |
| | Bis-Ethylhexyloxyphenol Methoxyphenyl | 4.00 | 4.00 |
| | Triazine | | |
| B | Aqua | qsp | qsp |
| | | 100% | 100% |
| | Glycerin | 2.00 | 2.00 |
| | Xanthan Gum | 0.20 | 0.20 |
| | Sodium EDTA | 0.20 | 0.20 |

Formulations Inv. 8 and Inv. 9 were manufactured as follows: Part A was heated to 80° C. under stirring without TiO2 and ZnO. When part A was homogenous TiO2 and ZnO were added, homogenised with ultra turrax device till TiO2 and ZnO were well dispersed and the part was homogeneous.

Part B was heated to 80° C. under stirring and added part A was added into part B under stirring, followed by homogenising with ultra turrax device.

The formulation was cooled down to room temperature under stirring.

TABLE 3
Stick formulations

| | | Formulation | Formulation |
|---|---|---|---|
| | INCI | Inv. 3 | Comp. 7 |
| A | Hydrogenated Castor Oil | 6.00 | 6.00 |
| | C12-15 Alkyl Benzoate | 15.00 | 15.00 |
| | Dicaprylyl Carbonate | 10.00 | 10.00 |
| | Dibutyl Adipate | 10.00 | 10.00 |
| | Hydrogenated Dimer | 1.00 | 1.00 |
| | Dilinoleyl/Dimethylcarbonate | | |
| | Copolymer | | |
| | Zinc Oxide (nano), | 10.00 | 10.00 |
| | Triethoxycaprylylsilane | | |
| | Titanium Dioxide (nano), Aluminum | 13.70 | 13.70 |
| | Hydroxide, Stearic Acid | | |
| | Bis-Ethylhexyloxyphenol | 5.00 | |
| | Methoxyphenyl Triazine | | |
| | Benzophenone -3 | | 5.00 |
| B | Cetearyl Alcohol | 5.00 | 5.00 |
| | Carnauba Cera | 6.00 | 6.00 |
| C | Talc | qsp 100% | qsp 100% |
| D | Undecane, Tridecane | 5.00 | 5.00 |

Formulations Inv. 3 and Comp. 7 were manufactured as follows: Part A was heated to 80° C. under stirring without TiO2 and ZnO. When part A was homogenous TiO2 and ZnO were added, homogenised via stirring till TiO2 and ZnO were well dispersed and the part was homogeneous.

Part B was added under stirring to 70° C., when homogeneous part C was added under stirring. Part D was added and the formulation was stirred. The formulation was filled up in the final container at 70° C.

TABLE 4
Formulations for antibacterial activity; Form. denotes Formulation

| | INCI | Form. A | Form. B | Form. C | Form. D | Form. E | Form. F |
|---|---|---|---|---|---|---|---|
| A | Polyglyceryl-2 | 1.00 | 1.00 | 1.00 | 1.00 | 1.00 | 1.00 |
| | Dipolyhydroxystearate | | | | | | |
| | Dicaprylyl | 3.00 | 3.00 | 3.00 | 3.00 | 3.00 | 3.00 |
| | Carbonate | | | | | | |
| | Dipropylheptyl | 3.00 | 3.00 | 3.00 | 3.00 | 3.00 | 3.00 |
| | Carbonate | | | | | | |
| | Caprylyl | 10.00 | 10.00 | 10.00 | 10.00 | 10.00 | 10.00 |
| | Caprylate/Caprate | | | | | | |
| | Bis- | | 1.00 | | | | |
| | Ethylhexyloxyphenol | | | | | | |
| | Methoxyphenyl | | | | | | |
| | Triazine | | | | | | |
| | Zinc Oxide (nano), | | | 1.00 | | | |
| | Triethoxycaprylylsilane | | | | | | |
| | Titanium Dioxide, | | | | 1.00 | | |
| | Aluminum | | | | | | |
| | Hydroxide, Stearic | | | | | | |
| | Acid | | | | | | |
| | Ethylhexyl | | | | | 1.00 | |
| | methoxycinnamate | | | | | | |
| | (OMC) | | | | | | |
| | Octocrylene (OCR) | | | | | | 1.00 |
| B | Water | Qsp | Qsp | Qsp | Qsp | Qsp | Qsp |
| | | 100% | 100% | 100% | 100% | 100% | 100% |
| | Glycerin | 5.00 | 5.00 | 5.00 | 5.00 | 5.00 | 5.00 |
| | Butylene Glycol | 10.00 | 10.00 | 10.00 | 10.00 | 10.00 | 10.00 |
| | Xanthan Gum | 2.00 | 2.00 | 2.00 | 2.00 | 2.00 | 2.00 |
| C | Laureth-7 Citrate | 0.50 | 0.50 | 0.50 | 0.50 | 0.50 | 0.50 |

Formulations A to F were manufactured as follows: The ingredients of part A, without Zinc oxide or Titanium dioxide for formulations C and D were weighted, and part A was heated to 80° C. with a magnetic stirring. When the temperature of A was 80° C., then Zinc oxide or Titanium dioxide for formulations C and D was added and homogenize with an Ultra Turrax type device.

The ingredients of part B without Xanthan Gum were weighted, then Xanthan Gum was added under stirring and heat to 80° C. When Part B was homogeneous, homogenize. Part C was added to part B under homogenization with an Ultra Turrax.

Part A was added into Part B/C under Ultra Turrax and homogenized further.

The formulations were cooled down under continuous stirring and adjusted to pH to >6.00 with a sodium hydroxide solution.

### Example 1—SPF of water/oil Formulations

The SPF in vitro values of the water/oil Formulations Inv. 1, Inv. 4, and Comp. 1 to Comp. 3 were measured after manufacturing and were compared. As can be seen from Table 5, Formulation Inv. 1 comprising TiO2, ZnO and BEMT provides a same or even higher SPF value, even though the total amount of UV filter is reduced when comparted to Comp. 2.

The addition of the organic BEMT enabled a better performance with a lower total concentration of UV filters. Formulation Inv. 4 comprising only TiO2 and BEMT provides a higher SPF value compared to e.g. Comp. 2 even though the total UV filter content is halved.

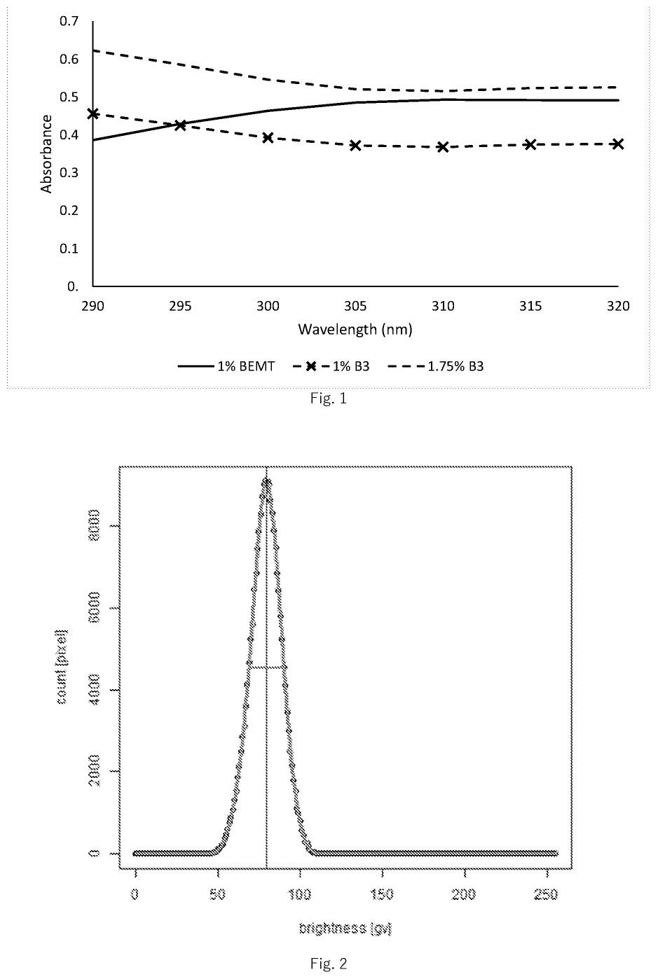

It is known that the broadband filter B3 has a lower efficacy than BEMT (see FIG. 1).

FIG. 1 depicts the spectra extracted from sunscreen simulator (https://sunscreensimulator.basf.com/Sunscreen_Simulator/computation) of the two lipophilic broad-spectrum filters BEMT and B3 at 1 wt.-% BEMT, at 1 wt.-% B3, and at 1.75 wt.-% B3. The simulation do not comprise an additional UV filter. The absorbance in the UVB range between 290 and 320 nm is depicted, which is mostly used when referring to the efficiency of a UV filter on the SPF value. As can be seen from the simulation in FIG. 1, the absorbance of 1% B3 is lower compared to the absorbance of the same amount (1%) of BEMT. It is needed 1.75 wt.-% B3 to achieve a similar or slightly higher absorbance than the absorbance of 1 wt.-% BEMT. However, even if the amount of B3 is increased by the factor 1.75 in the formulation (Comp. 3), the SPF still is lower than of Inv. 1 comprising a reduced amount of BEMT.

TABLE 5
Comparison of the water/oil Formulations Inv. 1, Inv. 4, and
Comp. 1 to Comp. 3; n.d. denotes “not determined”.

| | Inv. 1 | Comp. 1 | | Comp. 3 | |
|---|---|---|---|---|---|
| | (5% ZnO + | (5% ZnO + | Comp. 2 | (5% ZnO + | Inv. 4 |
| | 8.2% TiO2 + | 8.2% TiO2 + | (10% ZnO + | 8.2% TiO2 + | (6.8% TiO2 + |
| | 4% BEMT) | 4% B3) | 11.6% TiO2) | 7% B3) | 4% BEMT) |
| % UV | 17.2% | 17.2% | 21.6% | 20.2% | 10.8% |
| Filters | | | | | |
| (total) | | | | | |
| SPF in | 33 | 16 | 34 | 22 | 36 |
| vitro (plate | | | | | |
| method) | | | | | |
| UVA-PF in | 21 | n.d. | n.d. | n.d. | n.d. |
| vitro | | | | | |
| Critical | 380 | n.d. | n.d. | n.d. | n.d. |
| wavelength | | | | | |
| (nm) | | | | | |

### Example 2—SPF and Whitening of Oil/Water Formulations

The SPF in vitro values of the oil/water Formulations Inv. 2, Inv. 5, Inv. 6, Inv. 7, Comp. 4, Comp. 5, and Comp. 9 were measured after manufacturing and were compared. As can be seen from Table 6a, Formulation Inv. 2 comprising TiO2, ZnO and BEMT provides a higher SPF value, even though the total amount of UV filter is reduced when compared to Comp. 4 and Comp. 5.

Further, the SPF in vivo and in vitro values of Inv. 2 and Comp. 5 were compared. As can be seen from Table 6a, Inv. 2 comprising TiO2, ZnO and BEMT provides a higher SPF in vivo as well as in vitro value, even though the total amount of UV filter is reduced when comparted to Comp. 5. In addition, the SPF in vitro values of Inv. 2 and Comp. 4 were compared. As can be seen from Table 6a, the SPF value of Inv. 2 is higher even though less broadband UV filter is being needed. Inv. 6 comprising BEMT in the water phase provides an higher SPF in vitro compared to Comp. 4 and Comp. 5, even though the total UV filter concentration is reduced. Formulation Inv. 5 comprising only TiO2 and BEMT provides a higher SPF value compared to Comp. 4 and Comp. 5 even though the total UV filter content is reduced.

Table 6a further demonstrates that Inv. 7 comprising TiO2, ZnO and BEMT provides a higher SPF value when compared to Comp. 9 even though the total UV filter content is reduced.

Further, the whitening value of Inv. 2 and Comp. 5 were compared, wherein Formulation Inv. 2 provides a lower whitening value.

TABLE 6a
Comparison of the oil/water Formulations Inv. 2, Inv. 5, Inv. 6, Comp. 4, and
Comp. 5 (expected medium SPF formulations); n.d. denotes “not determined”.

| | | | | Inv. 6 | |
|---|---|---|---|---|---|
| | Inv. 2 | Comp. 4 | | (5.5% TiO2 + | |
| | (5.5% TiO2 + | (3% ZnO + | Comp. 5 | 3% ZnO + | Inv. 5 |
| | 3% ZnO + | 5.5% TiO2 + | (6.8% TiO2 + | 2.5% BEMT | (6.8% TiO2 + |
| | 2.5% BEMT) | 4.5% B3) | 10% ZnO) | aq. active) | 2.5% BEMT) |
| % Filters (total) | 11 | 13 | 16.8 | 11 | 9.3 |
| SPF in vitro | 14 | 11 | 7 | 16 | 21 |
| (plate method) | | | | | |
| UVA-PF in vitro | 12.5 | n.d. | n.d. | n.d. | n.d. |
| SPF in vivo | 11 | n.d. | 9 | n.d. | n.d. |
| Critical | 381 | n.d. | n.d. | n.d. | n.d. |
| wavelength (nm) | | | | | |
| Whitening delta L | 31 | n.d. | 33 | n.d. | n.d. |

TABLE 6b
Comparison of the oil/water Formulations Inv. 7 and Comp. 9 (expected
high SPF formulations); n.d. denotes “not determined”.

| | Inv. 7 (8.2% TiO2 + | Comp. 9 (11.6% TiO2 + |
|---|---|---|
| | 5% ZnO + 4% BEMT) | 10% ZnO) |
| % Filters (total) | 17.2 | 21.6 |
| SPF in vitro (plate | 26 | 18 |
| method) | | |

TABLE 6c
Formulations Inv. 8 and Inv. 9.

| | Inv. 8 | Inv. 9 |
|---|---|---|
| | (8.2% TiO2 + 5% ZnO + | (8.2% TiO2 + 5% |
| | 4% BEMT + Diethylhexyl | ZnO + 4% BEMT + |
| | 2,6-Naphthalate) | Polyester-8) |
| SPF in vitro | 45 | 50 |
| (plate method) | | |

### Example 3—SPF of Stick Formulations

The SPF in vitro values of the stick Formulations Inv. 3, Comp. 7 were compared. As can be seen from Table 7, Formulation Inv. 3 comprising TiO₂, ZnO and BEMT provides a higher SPF value.

TABLE 7
Comparison of the stick Formulations Inv. 3 and Comp. 7

| | Inv. 3 (10% ZnO + | Comp. 7 (10% ZnO + |
|---|---|---|
| | 13.7% TiO2 + 5% BEMT) | 13.7% TiO2 + 5% B3) |
| SPF in vitro | 119 | 73 |
| (plate method) | | |

### Example 4—Water Resistance

The in vitro water resistance of formulations Inv. 1, Comp. 2, and Comp. 3 was determined after manufacturing as above outlined. As can be seen from Table 8a, Formulation Inv. 1 comprising TiO2, ZnO and BEMT provides the best water resistance.

TABLE 8a
Comparison of water resistance of Inv. 1, Comp. 2, and Comp. 3

| | Inv. 1 (5% | Comp. 2 (10% | Comp. 3 (5% |
|---|---|---|---|
| | ZnO + 8.2% | ZnO + 11.6% | ZnO + 8.2% |
| | TiO2 + 4% BEMT) | TiO2) | TiO2 + 7% B3) |
| WR in vitro % | 65% | 50% | 40% |

The in vivo water resistance of formulations Inv. 2 and Comp. 5 was determined as above outlined. As can be seen from Table 8b, Formulation Inv. 2 comprising TiO2, ZnO and BEMT provides the best water resistance.

TABLE 8b
Comparison of water resistance of Inv. 2, Comp. 5

| | Inv. 2 (3% ZnO + | Comp. 5 (10% ZnO + |
|---|---|---|
| | 5.5% TiO2 + 2.5% BEMT) | 6.8% TiO2) |
| WR in vivo (40 | 99 | 76 |
| min, %) | | |

### Example 5—Stickiness

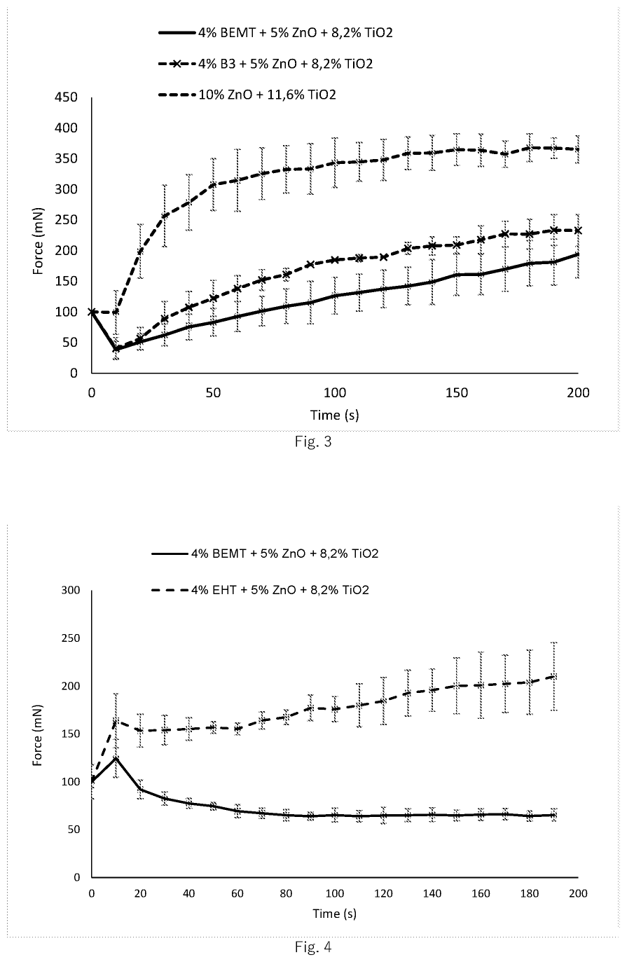

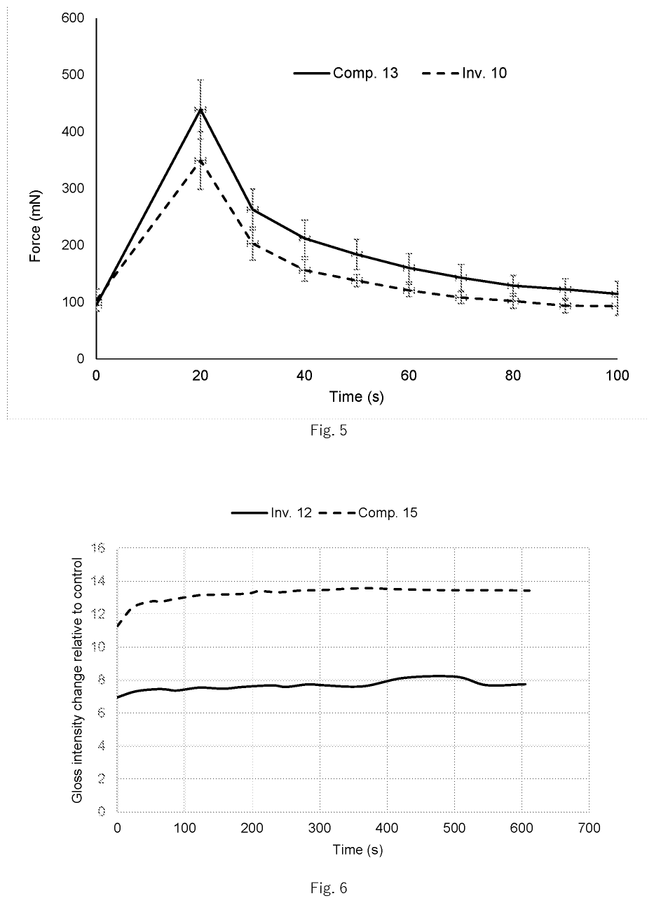

The stickiness of water/oil Formulations Inv. 1, Comp. 1, and Comp. 2 and oil/water Formulations Inv. 7, Inv. 10, Comp. 10, and Comp. 13 was evaluated after manufacturing according to the above-outlined method. As can be seen from FIG. 3 for water in oil formulations and FIG. 4 and FIG. 5 for oil in water formulations, Formulations Inv. 1, Inv. 7, and Inv. 10 comprising TiO2, ZnO and BEMT provide the lowest stickiness.

### Example 6—Film Homogeneity

The film homogeneity of oil/water formulations having an expected medium SPF Inv. 2, Inv. 5, Inv. 6, Comp. 5, Comp. 6, and oil/water formulations having an expected high SPF of Inv. 7 and Comp. 9, Comp. 10, and Comp. 11 and of water/oil formulations high SPF of Inv. 4 and Comp. 2 was determined after manufacturing according to the above-outlined method. As can be seen from Tables 9a to 9c, the inventive formulations (e.g. Inv. 2 (see also FIG. 2), Inv. 4, Inv. 5, Inv. 6, and Inv. 7) mostly provide the narrowest half-width-half-maximum (HWHM) value. Hence, Inv. 2, Inv. 4, Inv. 6, Inv. 7, and Inv. 11 have the highest film homogeneity.

In particular, when comparing Inv. 2, Inv. 5, and Inv. 6 with Comp. 5 and Comp. 6, it can be seen that for O/W formulations having a medium SPF value the inventive formulations provide the most narrow HWHM value.

When comparing Inv. 7 with Comp. 9, Comp. 10, and Comp. 11, it can be seen that for O/W formulations having a high SPF the HWHM of the inventive formulation is narrower.

When comparing Inv. 4 with Comp. 2, it can be seen that for W/O formulations having high SPF the HWHM of the inventive formulation is narrower.

TABLE 9a
Comparison of the film homogeneity of (expected medium SPF
formulations) Inv. 2, Inv. 5, Inv. 6, Comp. 5, and Comp. 6

| | | | Inv. 6 | | |
|---|---|---|---|---|---|
| | Inv. 2 | | (5.5% TiO2 + | | Comp. 6 |
| | (3% ZnO + | Inv. 5 | 3% ZnO + | Comp. 5 | (3% ZnO + |
| | 5.5% TiO2 + | (6.8% TiO2 + | 2.5% BEMT | (10% ZnO + | 5.5% TiO2 + |
| | 2.5% BEMT) | 2.5% BEMT) | aq. active) | 6.8% TiO2) | 2.5% B3) |
| HWHM | 13.4 +/− 1.3 | 6.5 +/− 0.4 | 5.7 +/− 0.3 | 18.9 +/− 3.5 | 19.7 +/− 1.5 |

TABLE 9b
Comparison of the film homogeneity (expected high SPF formulations)
of Inv. 7, Comp. 9, Comp. 10, and Comp. 11

| | Inv. 7 | | Comp. 10 | Comp. 11 |
|---|---|---|---|---|
| | (8.2% TiO2 + | Comp. 9 | (8.2% TiO2 + | (5% ZnO + |
| | 5% ZnO + | (11.6% TiO2 + | 5% ZnO + | 8.2% TiO2 + |
| | 4% BEMT) | 10% ZnO) | 4% EHT) | 4% OCR) |
| HWHM | 5.6 +/− 0.3 | 21.6 +/− 3.7 | 17.9 +/− 3.9 | 11.9 +/− 1.6 |

TABLE 9c
Comparison of the film homogeneity of water/oil formulations
(expected high SPF formulations) Inv. 4, Comp. 2

| | Inv. 4 | Comp. 2 |
|---|---|---|
| | (6.8% TiO2 + 4% BEMT) | (10% ZnO + 11.6% TiO2) |
| HWHM | 4.9 +/− 0.3 | 10.8 +/− 1.0 |

### Example 7—Antibacterial Activity to Evaluate the Microbial Compatibility

The antibacterial activity to evaluate the microbial compatibility of Formulations A to F was performed according to the above-outlined method. As can be seen from Table 10, Formulation B shows less antibacterial effect than Formulation C and is more human skin microbiome friendly. In addition, Table 10 demonstrates that Formulation B comprising BEMT is more human skin microbiome friendly than Formulations E and F comprising OMC or OCR.

TABLE 10
Comparison of the antibacterial activity

| | 0 h (calculated) | 2 h value | 4 h value | 24 h value |
|---|---|---|---|---|
| Samples | [cfu/ml] | [cfu/ml] | [cfu/ml] | [cfu/ml] |
| Placebo (no filter) | 5.8E+05 | 2.4E+05 | 1.2E+05 | 5.8E+02 |
| Formulation A | | | | |
| 1% BEMT | | 2.9E+05 | 3.1E+05 | 1.7E+04 |
| Formulation B | | | | |
| Placebo (no filter) | 1.0E+06 | 1.1E+05 | 5.8E+04 | 6.1E+02 |
| Formulation A | | | | |
| 1% ZnO | | 2.4E+04 | 20 | <10 |
| Formulation C | | | | |
| Placebo (no filter) | 6.2E+05 | 7.8E+04 | 4.1E+04 | 7.5E+03 |
| Formulation A | | | | |
| 1% TiO2 | | 1.6E+04 | 2.1E+04 | 6.6E+03 |
| Formulation D | | | | |
| 1% OMC | 5.8E+05 | | | 1.5E+03 |
| Formulation E | | | | |
| 1% OCR | 5.8E+05 | | | 1.1E+03 |
| Formulation F | | | | |

### Example 8—Photostability

The photostability of several formulations was tested after manufacturing.

The Formulations Inv. 1 to 3, Comp. 1, Comp. 4, and Comp. 7 were applied on roughened quartz plates (2 μl/cm²). Plates were irradiated using Atlas CPS device In total, four plates were prepared for each irradiation condition. After 50 MED (minimal erythemal dose) irradiation, each plate was rinsed off with tetrahydrofuran. The rinsing solution was further analyzed via HPLC to determine the recovery of the UV filters. The values are depicted in Table 11.

TABLE 11
recovery (%) of the organic UV filters after
irradiation comparison of the photostability

| Organic | 2.5% BEMT | 4% BEMT | 5% BEMT |
|---|---|---|---|
| UV | (Inv. 2) vs. 4.5% | (Inv. 1) vs. 4% | (Inv. 3) vs. 5% |
| filter | B3 (Comp. 4) | B3 (Comp. 1) | B3(Comp. 7) |
| BEMT | 100% | 98% | 107% |
| B3 | 71% | 84% | 75% |

### Example 9—Fabric Staining Before and/or after Washing Delta b*

The fabric staining of two formulations was measured after manufacturing before and after washing.

As can be seen in Table 12, Inv. 7 provides reduced staining than Comp. 12.

TABLE 12
fabric staining before and/or after washing
of UV filter containing formulations Inv. 7

| | Comp. 12 (5% ZnO + | Inv. 7 (5% ZnO + |
|---|---|---|
| | 8.2% TiO2 + | 8.2% TiO2 + |
| | 4% DHHB) | 4% BEMT) |
| Δb* (before washing) | 11.2 | 9.25 |
| Δb* (after washing) | 8 | 5.5 |

The values are an average of 4 measurements performed per textile piece and 2 textiles pieces.

### Example 10—Color (Yellowing) of the Formulation

The yellow tone of two formulations was measured after manufacturing. A b value positive indicates the presence of a yellow tone, the higher the b value, the more the yellow tone of the formulation, As can be seen in Table 13, Inv. 11 provides a less yellow color of the formulation than Comp. 14.

TABLE 13
Yellow tone of two formulations

| | Inv. 11 | Comp. 14 |
|---|---|---|
| b value | 7.5 | 8.3 |

The values are an average of 4 measurements.

### Example 11—Gloss of the Formulation

The gloss of two formulations was measured after manufacturing. As can be seen in FIG. 6, Inv. 12 provides less gloss than Comp. 15.

1.-23. (canceled)

24. Sunscreen or daily care composition comprising
(i) bis-ethylhexyloxyphenol methoxyphenyl triazine (INCI bis-ethylhexyloxyphenol methoxyphenyl triazine);
(ii) titanium dioxide; and
(iii) zinc oxide,
wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol](bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dione (avobenzone).

25. The sunscreen or daily care composition according to claim 24,
wherein the titanium dioxide has a number-average elementary particle diameter of less than 1000 nm and/or
wherein the zinc oxide has a number-average elementary particle diameter of less than 1000 nm.

26. The sunscreen or daily care composition according to claim 24, wherein the titanium dioxide is coated and/or
wherein the zinc oxide is uncoated or coated.

27. The sunscreen or daily care composition according to claim 24,
wherein the weight ratio of the sum of titanium dioxide and zinc oxide to the bis-ethylhexyloxyphenol methoxyphenyl triazine is in the range of 1:1 to 20:1 and/or wherein the weight ratio of the titanium dioxide to the zinc oxide is in the range of 4:1 to 1:3, and/or comprising titanium dioxide and zinc oxide in an amount of 1 to 35 wt.-%, based on the total amount of the sunscreen or daily care composition and/or
comprising a total of 5 to 40 wt.-% of UV filters, based on the total weight of the sunscreen or daily care composition.

28. The sunscreen or daily care composition according to claim 24,
wherein the bis-ethylhexyloxyphenol methoxyphenyl triazine (INCI bis-ethylhexyloxyphenol methoxyphenyl triazine) is added in the oil phase.

29. The sunscreen or daily care composition according to claim 24,
wherein the sunscreen or daily care composition comprises at least one further, different UV filter or
wherein the sunscreen or daily care composition does not comprise a further, different UV filter.

30. The sunscreen or daily care composition according to claim 24,
wherein the sunscreen or daily care composition comprises at least one compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, a fused ring cyanoacrylate derivative, polyester-25, and combinations thereof.

31. The sunscreen or daily care composition according to claim 24, comprising
(i) bis-ethylhexyloxyphenol methoxyphenyl triazine (INCI bis-ethylhexyloxyphenol methoxyphenyl triazine) in an amount of 1 to 10 wt.-%;
(ii) titanium dioxide in an amount of 2 to 20 wt.-%; and
(iii) zinc oxide in an amount of 1 to 20 wt.-%;
each based on the total weight of the sunscreen or daily care composition.

32. The sunscreen or daily care composition according to claim 24, providing a SPF of more than 15 or a SPF of more than 50 or a SPF of more than 90 and/or providing a photostability of more than 85%.

33. The sunscreen or daily care composition according to claim 24, providing a higher SPF value than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, the titanium dioxide, and the zinc oxide in equal amounts as the sunscreen or daily care composition according to claim 24 and/or providing a higher photostability than a comparison sunscreen or daily care composition comprising a different UV filter, instead of bis-ethylhexyloxyphenol methoxyphenyl triazine, wherein the comparison sunscreen or daily care composition comprises the different UV filter, the titanium dioxide, and the zinc oxide in equal amounts as the sunscreen or daily care composition according to claim 24.

34. Method of enhancing the photostability, and/or reducing the stickiness, and/or reducing the fabric staining before and/or after washing, and/or reducing the gloss, and/or reducing the formulation yellowing, and/or improving the water resistance, and/or improving the film homogeneity, and/or improving the compatibility with the microbiome of a sunscreen or daily care composition comprising titanium dioxide and zinc oxide by applying bis-ethylhexyloxyphenol methoxyphenyl triazine,
wherein the sunscreen or daily care composition is free of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol](bisoctrizole), and butyloctyl salicylate, and wherein if the sunscreen or daily care composition further comprises (RS)-2-Ethylhexyl (2E)-3-(4-methoxyphenyl)prop-2-enoate (octinoxate) said sunscreen or daily care composition is free of 1-(4-(1,1-dimethylethyl)phenyl)-3-(4-methoxyphenyl)propane-1,3-dione (avobenzone).

35. Sunscreen or daily care composition comprising
(i) bis-ethylhexyloxyphenol methoxyphenyl triazine (INCI bis-ethylhexyloxyphenol methoxyphenyl triazine);
(ii) titanium dioxide; and
(iii) zinc oxide,
wherein the sunscreen or daily care composition is free of ethylhexyl salicylate (octisalate), 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3), and wherein at least one of the inorganic UV filters (ii) titanium dioxide or (iii) zinc oxide is coated.

36. The sunscreen or daily care composition according to claim 35,
wherein the titanium dioxide has a number-average elementary particle diameter of less than 1000 nm and/or the zinc oxide has a number-average elementary particle diameter of less than 1000 nm.

37. The sunscreen or daily care composition according to claim 35,
wherein the titanium dioxide is coated and/or the zinc oxide is uncoated or coated.

38. The sunscreen or daily care composition according to claim 35,
wherein the weight ratio of the sum of titanium dioxide and zinc oxide to the bis-ethylhexyloxyphenol methoxyphenyl triazine is in the range of 1:1 to 20:1 and/or wherein the weight ratio of the titanium dioxide to the zinc oxide is in the range of 4:1 to 1:3, and/or
comprising titanium dioxide and zinc oxide in an amount of 1 to 35 wt.-%, based on the total amount of the sunscreen or daily care composition and/or comprising a total of 5 to 40 wt.-% of UV filters, based on the total weight of the sunscreen or daily care composition.

39. The sunscreen or daily care composition according to claim 35,
wherein the sunscreen or daily care composition comprises at least one further, different UV filter or wherein the sunscreen or daily care composition does not comprise a further, different UV filter.

40. The sunscreen or daily care composition according to claim 35,
wherein the sunscreen or daily care composition comprises at least one compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, a fused ring cyanoacrylate derivative, polyester-25, and combinations thereof.

41. The sunscreen or daily care composition according to claim 35, comprising
(i) bis-ethylhexyloxyphenol methoxyphenyl triazine (INCI bis-ethylhexyloxyphenol methoxyphenyl triazine) in an amount of 1 to 10 wt.-%;
(ii) titanium dioxide in an amount of 2 to 20 wt.-%; and
(iii) zinc oxide in an amount of 1 to 20 wt.-%;
each based on the total weight of the sunscreen or daily care composition.

42. The sunscreen or daily care composition according to claim 35 providing a SPF of more than 15 or a SPF of more than 50 or a SPF of more than 90 and/or providing a photostability of more than 85%.

43. The sunscreen or daily care composition according to claim 35, providing a higher SPF value than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to claim 35 and/or providing a higher photostability than a comparison sunscreen or daily care composition comprising a different UV filter, preferably a different UV broadband filter, instead of bis-ethylhexyloxyphenol methoxyphenyl triazine, wherein the comparison sunscreen or daily care composition comprises the different UV filter, preferably the different UV broadband filter, the titanium dioxide, and the zinc oxide in equal amounts as the sunscreen or daily care composition according to claim 35.

44. Method of enhancing the photostability, and/or reducing the stickiness, and/or reducing fabric staining before and/or after washing, and/or reducing the gloss, and/or reducing the formulation yellowing, and/or improving the water resistance, and/or improving the film homogeneity, and/or improving the compatibility with the microbiome of a sunscreen or daily care composition comprising titanium dioxide and zinc oxide by applying bis-ethylhexyloxyphenol methoxyphenyl triazine, wherein the sunscreen or daily care composition is free of ethylhexyl salicylate, 3,3,5-Trimethylcyclohexyl 2-hydroxybenzoate (INCI Homosalate), butyloctyl salicylate, (RS)-2-Ethylhexyl (2E)-3-(4-methoxyphenyl)prop-2-enoate (INCI ethylhexyl methoxycinnamate), 2-ethylhexyl 2-cyano-3,3-diphenylprop-2-enoate (INCI octocrylene), and 2-hydroxy-4-methoxybenzophenone (INCI benzophenone-3) and wherein at least one of the inorganic UV filters titanium dioxide or zinc oxide is coated.

45. A sunscreen of daily care composition comprising
(i) bis-ethylhexyloxyphenol methoxyphenyl triazine (INCI bis-ethylhexyloxyphenol methoxyphenyl triazine); and
(ii) titanium dioxide,
wherein the sunscreen of daily care composition is free of a further, additional UV filter.

46. A sunscreen of daily care composition comprising
(i) bis-ethylhexyloxyphenol methoxyphenyl triazine (INCI bis-ethylhexyloxyphenol methoxyphenyl triazine); and
(ii) zinc oxide,
wherein the sunscreen of daily care composition is free of a further, additional UV filter.
